(19) United States
(12) Patent Application Publication
(10) Pub. No.: US 2022/0196677 A1
(43) Pub. Date: Jun. 23, 2022

(54) KITS, COMPOSITIONS AND METHODS FOR EVALUATING IMMUNE SYSTEM STATUS

(71) Applicant: THE NATIONAL INSTITUTE FOR BIOTECHNOLOGY IN THE NEGEV LTD., Beer Sheva (IL)

(72) Inventors: Alon Monsonego, Nir-Banim (IL); Esti Yeger-Lotem, Omer (IL); Yehezqel Elyahu, Yoqne'am Illit (IL); Idan Hekselman, Ramat Gan (IL)

(21) Appl. No.: 17/430,304

(22) PCT Filed: Mar. 2, 2020
(86) PCT No.: PCT/IL2020/050235

§ 371 Date: Aug. 11, 2021

Related U.S. Application Data
(60) Provisional application No. 62/813,235, filed on Mar. 4, 2019.

(51) Int. Cl. G01N 33/68; A61P 37/02

(52) U.S. Cl. CPC G01N 33/6893; G01N 2800/60; G01N 2800/52; A61P 37/02

(57) ABSTRACT

The present disclosure generally relates to the field of evaluating a status, in particular, an age of an immune system based on a level of biomarkers, the biomarkers indicative of CD4 T cell phenotype, specifically for identifying levels of CD4 cytotoxic cells and/or activated regulatory (a Treg) CD4 T cells and thereby identify diseases and disorders, such as, systemic inflammation.

### FIELD OF INVENTION

The present disclosure generally relates to the field of evaluating a status, in particular, an age of an immune system based on a level of biomarkers, the biomarkers indicative of CD4 T cell phenotype, specifically for identifying levels of CD4 cytotoxic cells and/or activated regulatory (aTreg) CD4 T cells and thereby identify diseases and disorders, such as, systemic inflammation.

### BACKGROUND

One of the key hallmarks of aging is the deterioration of the immune system, rendering the elderly more prone to infections, chronic inflammatory disorders, and vaccination failure. A significant change observed in aging relates to the composition and functionally of CD4 T cells, the main orchestrators of adaptive immune responses. In young rodents and humans, CD4 T cells comprise a high frequency of naïve cells, reflecting the ability of the immune system to encounter new antigens, respond to them effectively, and generate immune memory. With aging, this naïve subset shrinks along with the accumulation of highly differentiated memory cells which often shows dysregulated properties. For example, previous studies performed on classical CD4 T-cell subsets described reduced proliferation, lower secretion of IL-2 cytokine and accumulation of metabolic defects, along with higher production of pro-inflammatory mediators. Furthermore, regulatory T cells (Tregs) appear to accumulate with age and further contribute to reduced responsiveness of effector T cells. These changes are assumed to result from age-related thymus involution, repeated antigen encounters and intrinsic cellular senescence processes. In addition, systemic low-grade chronic inflammation that develops with age, referred to as “inflammaging”, also appears to impact the phenotype and function of CD4 T cells.

There remains an unmet need to identify alterations in immune cell populations and provide age-associated assays for improving the functionality of subject's immune system and for preventing, halting and/or attenuating diseases and disorders associated with age-related immune conditions, preferably in a personalized manner.

### SUMMARY

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

Provided herein are biomarkers, compositions, kits and methods for assessing an immunological age of an immune system and use thereof for improving treatment efficiency by providing custom-made and/or personalized treatment designed to perfectly fit the treatment protocol to the immunological age of an immune system. Surprisingly, two new subsets of CD4 T-cells were found to accumulate with age: one subset exhibits an activated phenotype of regulatory T cells (aTregs) and another with a cytotoxic and pro-inflammatory expression profile (CD4 cytotoxic T lymphocytes, CTLs; e.g. FIG. 2D and Table 1). Notably, a preliminary study in human subjects revealed that CD4 CTLs accumulate also in elderly healthy human individuals (FIGS. 6A-6C). Furthermore, it has been found that the transcription factors (TFs) required for the CD8 CTL lineage are active in the CD4 CTLs, and that the in vitro stimulation of these cells results in a significantly higher expression of pro-inflammatory and cytotoxic molecules, as compared with EM and exhausted T cells (e.g. FIG. 3D). Although cytotoxicity was generally thought to be irreversibly blocked in the CD4 lineage, this findings is advantageous as it indicate that the fate of CD4 T helper (Th) cells can change and lead to a post-thymic differentiation of MHCII-restricted cytotoxic CD4 T cells with killing functions, similar to the CD8 CTL lineage.

In some embodiments, there is provided a method for evaluating an immunological age of an immune system of a subject, the method comprising:
a) obtaining a biological sample from a subject, the biological sample comprising biomarkers associated with one or more subsets of CD4 T cells;
b) detecting a level of a plurality of the biomarkers, thereby identifying the presence of one or more subsets of CD4 T cells in the immune system, wherein the one or more subsets of CD4 T cells comprise at least one of cytotoxic T cells and activated regulatory (aTreg) CD4 T cells; and
c) evaluating the immunological age of the immune system based on the presence of the plurality of biomarkers identified in the biological sample.

In some embodiments, said identifying the presence of one or more subsets of CD4 T cells comprises determining the amount of each identified subset of CD4 T cells relative to a control value.

In some embodiments, the one or more subsets of CD4 T cells further comprise at least one of naïve CD4 T cells, naïve_Isg15 CD4 T cells, effector memory (TEM) CD4 T cells, exhausted CD4 T cell and, rTregs CD4 T cells.

In some embodiments, said evaluating the immunological age of the immune system comprises evaluating the presence of each subset of CD4 T cells in the biological sample based on the level of the plurality of biomarkers associated therewith, comparing the level of the plurality of biomarkers to mature threshold value, wherein detecting a high probability score for said level with respect to the mature threshold value indicates that the immunological age of the immune system is mature.

In some embodiments, said evaluating the immunological age of the immune system comprises evaluating the presence of each subset of CD4 T cells in the biological sample based on the level of the plurality of biomarkers associated therewith, comparing the level of the plurality of biomarkers to young threshold value, wherein detecting a high probability score for said level with respect to the young threshold value indicates that the immunological age of the immune system is young.

In some embodiments, the one or more subsets of CD4 T cells is naïve CD4 T cells and the presence of the naïve CD4 T cells subset is above 50%, thereby indicating that the immunological age of the immune system is young.

In some embodiments, the one or more subsets of CD4 T cells is naïve CD4 T cells and the presence of the naïve CD4 T cells subset is below 50%, thereby indicating that the immunological age of the immune system is mature.

In some embodiments, the one or more subsets of CD4 T cells is cytotoxic CD4 T cells and the presence of the cytotoxic CD4 T cells subset is above 1%, thereby indicating that the immunological age of the immune system is mature.

In some embodiments, the one or more subsets of CD4 T cells is exhausted CD4 T cells and the presence of the exhausted CD4 T cells subset is above 1%, thereby indicating that the immunological age of the immune system is mature.

In some embodiments, the one or more subsets of CD4 T cells is aTregs CD4 T cells and the presence of the aTregs CD4 T cells subset is above 1%, thereby indicating that the immunological age of the immune system is mature.

In some embodiments, the one or more subsets of CD4 T cells is TEM CD4 T cells and the presence of the TEM CD4 T cells subset is above 4%, thereby indicating that the immunological age of the immune system is mature.

In some embodiments, the one or more subsets of CD4 T cells is selected from TEM CD4 T cells, cytotoxic CD4 T cells, exhausted CD4 T cells and aTregs CD4 T cells and the presence of said one or more subsets of CD4 T cells is above 9%, thereby indicating that the immunological age of the immune system is mature.

In some embodiments, the plurality of biomarkers is selected from the group consisting of: EOMES, CCL3, CCL4, CCLS, CCR7, CD7, CD8, CD137, CD134, CD25, CD44, CD62L, CD74, CD81, CD200, Cst7, Ms4a4b, NKG2D, Nfatc1, Runx2, Runx3, Tbx21, GzmB, GzmK, perforin, FOXP3, GITR, Helios, Lgalsl, IGFbp4, LAG3, IL-1a, IL-1b, IL1R2, IL2RA, IL-6, IL-10, IL-17A, IL-21, IL-18R1, IL-27, IFN-b, IFN-g, Isg15, PD1, Lefl, Lfit3, MCP1, Satbl, Ccr7, Aw112010, S100a11, S100a10, S100a4, Sell, Pdcdl, Izumolr, Ikzf2, Igfbp4, Itgbl, Itgb7, GM-CSF, Sostdcl, Tbc1d4, TNF, TNFRSF4, TNFRSF8, TNFSF8, Ctla2a, Ctla4 and TNF-RSF9/4.

In some embodiments, the plurality of biomarkers comprises at least three biomarkers.

In some embodiments, the plurality of biomarkers is selected from CD137, CD134, FOXP3+, GITR+, Helios+, CD74, HLA-DR, and wherein the one or more subsets of CD4 T cells identified in the biological sample is aTreg CD4 T cell.

In some embodiments, wherein the plurality of biomarkers is selected from Nkg7, Runx3, Eomes, Gzmk, IFN-b, IL-27, IL21, IL 17A, Cc13, Cc14 and Cc15, and wherein the one or more subsets of CD4 T cells identified in the biological sample is CD4 cytotoxic cells.

In some embodiments, the plurality of biomarkers is selected from Cd200, Lag3, Hif1a, Nfatc1 and Pdcd1, and wherein the one or more subsets of CD4 T cells identified in the biological sample is exhausted CD4 T cells.

In some embodiments, the plurality of biomarkers comprises at least one of Itgb7 and IL-18R1, and wherein the one or more subsets of CD4 T cells identified in the biological sample is TEM CD4 T cells.

In some embodiments, the plurality of biomarkers comprises at least one of Ms4a4b and Sell, and wherein the one or more subsets of CD4 T cells identified in the biological sample is rTregs CD4 T cells.

In some embodiments, the biological sample comprises serum.

In some embodiments, the biological sample comprises CD4 T cells and the method further comprising isolating a CD4 T cell population from the biological sample.

In some embodiments, there is provided a kit for evaluating an immunological age of an immune system of a subject, the kit comprising:
at least two biomarker identifiers configured to determine a level of at least two biomarkers in a biological sample, wherein the at least two biomarkers are associated with one or more subsets of CD4 T cells, and wherein the one or more subsets of CD4 T cells comprise CD4 cytotoxic cells and/or activated regulatory (aTreg) CD4 T cells; and
instructions for use.

The In some embodiments, the kit further comprises at least one sampling tube configured to receive the biological sample.

In some embodiments, there is provided a method for treating an age-related frailty in a subject in need thereof, the method comprising:
obtaining a biological sample from a subject comprising biomarkers associated with one or more subsets of CD4 T cells;
detecting a level of a plurality of the biomarkers thereby identifying the presence of one or more subsets of CD4 T cells in the immune system, wherein the one or more subsets of CD4 T cells comprise at least one of CD4 cytotoxic cells and activated regulatory (aTreg) CD4 T cells;
identifying an immunological age of the immune system based on the presence of the plurality of biomarkers identified in the biological sample; and
administering to the subject one or more therapeutics, based on the identified immunological age of the immune system.

In some embodiments, said identifying the one or more subsets of CD4 T cells comprises determining the amount of each identified subset of CD4 T cells, relative to a control value.

In some embodiments, the one or more subsets of CD4 T cells further comprise at least one of naïve CD4 T cells, naïve_Isg15 CD4 T cells, effector memory (TEM) CD4 T cells, exhausted CD4 T cell and rTregs CD4 T cells.

In some embodiments, said evaluating the immunological age of the immune system comprises evaluating the presence of each subset of CD4 T cells in the biological sample based on the level of the plurality of biomarkers associated therewith, comparing the level of the plurality of biomarkers to mature threshold value, wherein detecting a high probability score for said level with respect to the mature threshold value indicates that the immunological age of the immune system is mature.

In some embodiments, said evaluating the immunological age of the immune system comprises evaluating the presence of each subset of CD4 T cells in the biological sample based on the level of the plurality of biomarkers associated therewith, comparing the level of the plurality of biomarkers to young threshold value, wherein detecting a high probability score for said level with respect to the young threshold value indicates that the immunological age of the immune system is young.

In some embodiments, the one or more subsets of CD4 T cells is naïve CD4 T cells and the presence of the naïve CD4 T cells subset is above 50%, thereby indicating that the immunological age of the immune system is young.

In some embodiments, the one or more subsets of CD4 T cells is naïve CD4 T cells and the presence of the naïve CD4 T cells subset is below 50%, thereby indicating that the immunological age of the immune system is mature.

In some embodiments, the one or more subsets of CD4 T cells is cytotoxic CD4 T cells and the presence of the cytotoxic CD4 T cells subset is above 1%, thereby indicating that the immunological age of the immune system is mature.

In some embodiments, the one or more subsets of CD4 T cells is exhausted CD4 T cells and the presence of the exhausted CD4 T cells subset is above 1%, thereby indicating that the immunological age of the immune system is mature.

In some embodiments, the one or more subsets of CD4 T cells is aTregs CD4 T cells and the presence of the aTregs CD4 T cells subset is above 1%, thereby indicating that the immunological age of the immune system is mature.

In some embodiments, the one or more subsets of CD4 T cells is TEM CD4 T cells and the presence of the TEM CD4 T cells subset is above 4%, thereby indicating that the immunological age of the immune system is mature.

In some embodiments, the one or more subsets of CD4 T cells is selected from TEM CD4 T cells, cytotoxic CD4 T cells, exhausted CD4 T cells and aTregs CD4 T cells and the presence of said one or more subsets of CD4 T cells is above 9%, thereby indicating that the immunological age of the immune system is mature.

In some embodiments, the plurality of biomarkers is selected from the group consisting of: EOMES, CCL3, CCL4, CCL5, CCR7, CD7, CD8, CD137, CD134, CD25, CD44, CD62L, CD74, CD81, CD200, Cst7, Ms4a4b, NKG2D, Nfatcl, Runx2, Runx3, Tbx21, GzmB, GzmK, perforin, FOXP3, GITR, Helios, Lgals1, IGFbp4, LAG3, IL-1a, IL-1b, IL1R2, IL2RA, IL-6, IL-10, IL-17A, IL-21, IL-18R1, IL-27, IFN-b, IFN-g, Isg15, PD1, Lefl, Lfit3, MCP1, Satb1, Ccr7, Aw112010, S100a11, S100a10, S100a4, Sell, Pdcd1, Izumo1r, Ikzf2, Igfbp4, Itgb1, Itgb7, GM-CSF, Sostdc1, Tbc1d4, TNF, TNFRSF4, TNFRSF8, TNFSF8, Ctla2a, Ctla4 and TNF-RSF9/4.

In some embodiments, the one or more therapeutics is an agent targeting at least one of the plurality of biomarkers. In some embodiments, the agent is selected from the group consisting of an antibody, a siRNA, a microRNA, a small molecule or any combination thereof.

In some embodiments, the antibody is an NKG2D antibody, a CD7 antibody, a CD134 antibody, a CD137 antibody, a GITR antibody or any combination thereof.

In some embodiments, the one or more therapeutics is an age-related drug.

In some embodiments, the age-related drug is a statin, an aspirin or a combination thereof.

In some embodiments, the age-related frailty is an immune-associated disease.

In some embodiments, the immune-associated disease is chronic inflammation or cancer.

In some embodiments, the immune-associated disease is immune deficiency.

In some embodiments, there is provided a composition for use in the treatment of an age-related frailty, the composition comprising a plurality of biomarker identifiers configured to determine a level of a plurality of biomarkers associated with subsets of CD4 T cells in a biological sample or an isolate thereof thereby evaluate the presence of one or more subsets of CD4 T cells in the immune system corresponding to the biological sample, wherein the one or more subsets of CD4 T cells comprise CD4 cytotoxic cells and/or activated regulatory (aTreg) CD4 T cells and wherein said treatment is based on the identified immunological age of the immune system.

In some embodiments, the biomarker identifiers comprise a plurality of oligonucleotides, each capable of hybridizing to at least one biomarker in the plurality of biomarkers.

In some embodiments, the biomarker identifiers comprise a plurality of nucleotide primer pairs each adjusted to flank at least one biomarker in the plurality of biomarkers.

In some embodiments, said determine a level of a plurality of biomarkers associated with subsets of CD4 T cells in a biological sample comprises determine, based on the level of the plurality of biomarkers, the relative amount of each identified subset from the one or more subsets of CD4 T cells, compared to a control value.

In some embodiments, the one or more subsets of CD4 T cells further comprise at least one of naïve CD4 T cells, naïve_Isg15 CD4 T cells, effector memory (TEM) CD4 T cells, exhausted CD4 T cell and, rTregs CD4 T cells.

In some embodiments, said evaluate the immunological age of the immune system comprises evaluate the presence of each subset of CD4 T cells in the biological sample based on the level of the plurality of biomarkers associated therewith, compare the level of the plurality of biomarkers to mature threshold value, wherein a high probability score for said level with respect to the mature threshold value indicates that the immunological age of the immune system is mature.

In some embodiments, said evaluate the immunological age of the immune system comprises evaluate the presence of each subset of CD4 T cells in the biological sample based on the level of the plurality of biomarkers associated therewith, compare the level of the plurality of biomarkers to young threshold value, wherein a high probability score for said level with respect to the mature threshold value indicates that the immunological age of the immune system is young.

In some embodiments, the one or more subsets of CD4 T cells is naïve CD4 T cells when the presence of the naïve CD4 T cells subset is below 50%, thereby indicating that the immunological age of the immune system is mature. In some embodiments, the one or more subsets of CD4 T cells is cytotoxic CD4 T cells when the presence of the cytotoxic CD4 T cells subset is above 1%, thereby indicating that the immunological age of the immune system is mature. In some embodiments, the one or more subsets of CD4 T cells is exhausted CD4 T cells when the presence of the exhausted CD4 T cells subset is above 1%, thereby indicating that the immunological age of the immune system is mature. In some embodiments, the one or more subsets of CD4 T cells is aTregs CD4 T cells when the presence of the aTregs CD4 T cells subset is above 1%, thereby indicating that the immunological age of the immune system is mature. In some embodiments, the one or more subsets of CD4 T cells is TEM CD4 T cells when the presence of the TEM CD4 T cells subset is above 4%, thereby indicating that the immunological age of the immune system is mature. In some embodiments, the one or more subsets of CD4 T cells is selected from TEM CD4 T cells, cytotoxic CD4 T cells, exhausted CD4 T cells and aTregs CD4 T cells when the presence of said one or more subsets of CD4 T cells is above 9%, thereby indicating that the immunological age of the immune system is mature.

In some embodiments, the plurality of biomarkers is selected from the group consisting of: EOMES, CCL3, CCL4, CCL5, CCR7, CD7, CD8, CD137, CD134, CD25, CD44, CD62L, CD74, CD81, CD200, Cst7, Ms4a4b, NKG2D, Nfatc1, Runx2, Runx3, Tbx21, GzmB, GzmK, perforin, FOXP3, GITR, Helios, Lgals1, IGFbp4, LAG3, IL-1a, IL-1b, IL1R2, IL2RA, IL-6, IL-10, IL-17A, IL-21, IL-18R1, IL-27, IFN-b, IFN-g, Isg15, PD1, Lef1, Lfit3, MCP1, Satb1, Ccr7, Aw112010, S100a11, S100a10, S100a4, Sell, Pdcd1, Izumo1r, Ikzf2, Igfbp4, Itgb1, Itgb7, GM-CSF, Sostdc1, Tbc1d4, TNF, TNFRSF4, TNFRSF8, TNFSF8, Ctla2a, Ctla4 and TNF-RSF9/4.

In some embodiments, said treatment comprises an agent targeting at least one of the plurality of biomarkers. In some embodiments, the agent is selected from the group consisting of an antibody, a siRNA, a microRNA, a small molecule or any combination thereof.

In some embodiments, the antibody is an NKG2D antibody, a CD7 antibody, a CD134 antibody, a CD137 antibody, a GITR antibody or any combination thereof.

In some embodiments, said treatment comprises an age-related drug. In some embodiments, the age-related drug is a statin, an aspirin or a combination thereof. In some embodiments, the age-related frailty is an immune-associated disease.

In some embodiments, the immune-associated disease is selected from Alzheimer's disease, depression, short-term memory loss, urinary incontinence, osteoporosis, osteoarthritis, Parkinson's disease, malignant diseases, hypertension, cardiovascular disease, amyotrophic lateral sclerosis (ALS), heart diseases, diabetes, chronic inflammation, inflammation, systemic inflammation, dementia, neurodegenerative diseases, cellular immune dysfunction and obesity.

In some embodiments, the immune-associated disease is cancer. In some embodiments, the immune-associated disease is immune deficiency.

In some embodiments, there is provided a method for determining the severity of an immune system dysregulation in a subject in need thereof, the method comprising:
a) obtaining a biological sample from a subject, the biological sample comprising biomarkers associated with one or more subsets of CD4 T cells and/or serum from the subject;
b) detecting the presence of at least one biomarker associated with at least one subset of CD4 T cells selected from aTreg CD4 T cells, CD4 cytotoxic T cells and exhausted CD4 T cells; and
c) identifying the immune system as dysregulated immune system based on the presence of the at least one biomarker which deviates from regulated immune system control.

In some embodiments, the presence of the at least one biomarker which deviates from regulated immune system control corresponds to a level of CD4 cytotoxic T cells above the regulated immune system control.

In some embodiments, the presence of the at least one biomarker which deviates from regulated immune system control corresponds to a level of aTreg CD4 cells above the regulated immune system control.

In some embodiments, the at least one biomarker comprises a plurality biomarkers.

In some embodiments, the at least one biomarker is selected from GzmB, Perforin, IL-10 and TGF-beta.

In some embodiments, there is provided a pharmaceutical composition for the treatment of age-related frailty, wherein the age-related frailty is determined based on the presence of a plurality of biomarkers, identified in a biological sample, the plurality of the biomarkers is associated with one or more subsets of CD4 T cells in an immune system corresponding to the biological sample, wherein the plurality of biomarkers indicate the presence of subsets of CD4 T cells comprising at least one of cytotoxic T cells and activated regulatory (aTreg) CD4 T cells, and wherein said pharmaceutical composition comprises a therapeutically effective agent for the treatment of age-related frailty.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures.

FIG. 1A is a schematic presentation of the experimental procedure used for obtaining gene expression signatures of CD4 T cells in young and old mice.

FIG. 1B shows representative flow cytometry plots of highly pure CD4⁺TCRβ⁺ T cells after magnetic enrichment and sorting, discarding CD8⁺CD19⁺CD11b⁺NK1.1⁺ cells.

FIG. 1C shows representative analysis of the sorted young and old CD4 T cells stained for CD44 and CD62L surface markers (unpaired T test, ****p<10⁻⁴).

FIG. 1D shows representative T-SNE projections of CD4 T cells including 13,186 and 10,821 cells from young (turquoise/gray) and old (brown/black) mice, respectively (each dot represents a single cell).

FIG. 1E shows representative scatter plot exemplifying differentially expressed genes between age groups (each dot represents a gene, with significantly upregulated genes (log(fold-change)>0.2, adjusted p<10⁻³) in young (turquoise/gray, right side) and old mice (brown/black, left side).

FIGS. 1F and 1G show T-SNE projections with cells colored by the expression levels of age marker genes. Markers were selected as highly expressed genes within an age group (log(fold-change)>0.4) that best distinguish between age groups according to a ROC analysis (F, AUC>0.61, power>0.23; G, AUC>0.66, power>0.33).

FIGS. 1H shows mean and standard deviations (SDs) of grip test (Y axis=sxg) and hanging test (Y axis=Newtons).

FIG. 1I shows mean and SDs of food intake, water intake, total activity, and wheel activity (n=6-8) in old mice (each dot represents one mouse).

FIG. 1J shows correlations between PBMC counts in the spleen and grip test (n=12; r=0.09, P=0.404).

FIG. 1K is a heatmap showing the correlation between the frequency of CD4 T cell subsets (naïve, exhausted, memory, and CD4 CTLs) and the physical and metabolic tests (n=6-12) calculated assuming the data exhibit a Gaussian distribution (Pearson correlation).

FIG. 1L shows representative flow cytometry plots presenting the gating strategy of CD62L-CD44+ effector memory (EM) and CD62L+CD44− naïve CD4 T cells and the percentage of EOMES+CCL5+CD4 CTLs in each subset.

FIG. 1M shows quantitative analysis of CD4 CTL frequencies among CD4 T cells in a cohort of 20-month-old mice (n=5; paired Student t test, P=0.014).

FIG. 2A shows a T-SNE projection of all 24,007 CD4 T cells presenting seven different subsets, identified via SNN modularity optimization-based clustering algorithm, followed by integration of similar clusters.

FIG. 2B shows a heatmap of gene expression z-scores across cells.

FIG. 2C shows violin plots presenting the expression (z-score) of selected canonical marker genes across all seven subsets.

FIG. 2D shows pie charts presenting the percentage of cells belonging to each of the seven subsets in a young mouse and an old mouse.

FIG. 2E shows the log odds ratio between the frequency of each subset in old versus young mice, across all pairs, following computed enrichment.

FIG. 2F is a heatmap showing Spearman's rank correlation coefficient (Rho) between the frequency of each subset and cytokines concentration (μg/ml) measured in serum of all young and old mice (P values were adjusted using the Benjamini-Hochberg procedure (α<0.05)).

FIG. 2G. is a scheme illustrating the major changes that occur in the population of CD4 T cells during aging.

FIGS. 3A-3C show representative scatter plots presenting differentially expressed genes between cytotoxic and TEM cells, exhausted and TEM cells, and aTregs and rTregs cells, respectively, where only cells from old mice were considered in the analysis and each dot represents a gene, with significantly upregulated genes (log(fold-change)>0.2, p<10⁻³) colored by their corresponding subset.

FIG. 3D shows representative t-SNE plots of CD4 T cells obtained from young (top left panel) and old (bottom left panel) mice and mean fluorescence intensities (MFIs) of each marker (right panels) obtained by analysis based on the expression of 10 marker proteins chosen according to the gene expression profiles.

FIG. 3E shows representative flow cytometry plots gated on FOXP3⁺ cells presenting the MFI of selected marker proteins that relate to Tregs activation, projected on CD44^{low}CD81⁻ (rTregs) and CD44⁺CD81⁻ (aTregs) populations.

FIG. 3F shows bar plots showing quantitatively the MFIs of each marker in rTregs and aTregs. Each dot represents a mouse (n=6, from two different experiments; Paired T test, *p<0.05, **p<0.01, ***p<10⁻³).

FIG. 4A shows representative flow cytometry plots (left) and corresponding analysis (right) presenting the prevalence of exhausted cells defined as PD1⁺CD62L⁻ cells (pink/positive slope; R=0.94) and naïve cells, defined as CD62L⁺PD1⁻ out of CD4⁺EOMES⁻CCL5⁻FOX3⁻ cells (blue/negative slop; R=0.96) measured via flow cytometry in mice at the age of 2 and 16 months, where shaded areas of each graph (right) represent ±s.e.m.

FIG. 4B shows representative flow cytometry plots (left) and corresponding analysis (right) presenting the prevalence of aTregs cells defined as CD81⁺CD44⁺ cells out of CD4⁺FOXP3⁺CD8⁻ cells (R=0.78) measured via flow cytometry in mice at the age of 2 and 16 months, where shaded areas the graph represents ±sem.

FIG. 4C shows representative flow cytometry plots (left) and corresponding analysis (right) presenting the prevalence of cytotoxic cells defined as EOMES⁺CCL5⁺ out of CD4⁺CD8⁻ cells (R=0.91) measured via flow cytometry in mice at the age of 2 and 24 months, where shaded areas the graph represents ±sem.

FIG. 4D shows the percentages of exhausted cells in various immune sites. Bars colored similarly correspond to the same mouse. Data include three different experiments, n=2-3 per experiment. P values computed via one-way ANOVA test with Tukey correction for multiple comparisons (*p<0.05, **p<0.01, ***p<10⁻³).

FIG. 4E shows the percentages of aTregs cells in various immune sites. Bars colored similarly correspond to the same mouse. Data include three different experiments, n=2-3 per experiment. P values computed via one-way ANOVA test with Tukey correction for multiple comparisons (*p<0.05, **p<0.01, ***p<10⁻³).

FIG. 4F shows the percentages of cytotoxic cells in various immune sites. Bars colored similarly correspond to the same mouse. Data include three different experiments, n=2-3 per experiment. P values computed via one-way ANOVA test with Tukey correction for multiple comparisons (*p<0.05, **p<0.01, ***p<10⁻³).

FIG. 5A is heatmap of old CD4 T cells showing 27 high-confidence regulons that were active consistently across all old mice, where active regulons per cell appear in black, horizontal color bar indicates the subset associated with each cell.

FIG. 5B is a radared-balloon plot showing regulons' activity per subset per mouse, each circle corresponds to a single subset and is divided into four slices, one per mouse, where slice size reflects the fraction of subset cells with active regulon, normalized to the maximal fraction of that regulon across mice and subsets.

FIG. 5C shows representative flow cytometry plots (left panel) of sorted CD25^{high}CD81⁻ (yellow/rTregs) and CD25^{high}CD81⁺ (brown/aTregs) and corresponding bar plot (right panel) presenting the suppression ability (%) of rTregs versus aTregs (each dot represents a mouse (n=8, from two independent experiments; unpaired T test (*p<0.05).

FIG. 5D shows the percentage of cells positive to pro-inflammatory and cytotoxic cytokines in TEM, exhausted and cytotoxic subsets after 48 hours of activation (lines connect measurements within the same mouse; data from two independent experiments, n=7 mice; paired T test (*p<0.05, **p<0.01, ***p<10⁻³, ****p<10⁻⁴).

FIG. 5E is a schematic illustration of the accumulation of RECs with age, showing their key transcription factors and top markers (within and to the right of each cell, respectively), which point to a dysregulated immune response.

FIG. 6A shows the relative frequency of total CD4 T cells in peripheral blood mononuclear cell (PBMC) obtained from young and old healthy human subjects.

FIG. 6B shows the relative frequency of naïve CD4 T cells (Sell) in peripheral blood mononuclear cell (PBMC) obtained from young and old healthy human subjects.

FIG. 6C shows the relative frequency of CD4 cytotoxic T cells (CTLs) in peripheral blood mononuclear cell (PBMC) obtained from young and old healthy human subjects.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The complexity of the immune system, and, particularly, the large variety of the cells comprising it, renders its investigation challenging. Until recently, the various cell lineages of the immune system were explored and traced either by using known cell markers or by analyzing bulk populations; however, these analyses cannot detect small populations of cells or novel subtypes of cells whose markers are yet unknown. These difficulties may be addressed by the single-cell RNA-sequencing (scRNA-seq) technology—a technology that provides RNA-sequencing profiles for hundreds and even thousands of single cells, which are then characterized and clustered in an unbiased manner Each cluster can be associated with a potentially new marker gene, and the population structure can be assessed at a larger scale. For example, applying this technology, lymphopoiesis was shown to be decreased with age and CD4 T cells were shown to demonstrate a higher cell-to-cell variability in the expression of core activation programs in older ages.

### Definitions

As used herein the term “immunological age” of an immune system refers to a profile of an immune system, specifically its CD4 T cell profile and optionally also the pattern of serum inflammatory markers (chemokines and cytokines) typical for subjects of a certain age. It is understood that there may be a discrepancy between a subject's chronological age and the immunological age of his/her immune system. According to some embodiments, the immunological age is identified as old, dysregulated, aged, mature and/or exhausted. Alternatively, the immunological age is identified as young, regulated, efficient and/or naïve.

As used herein, the term “old” when referring to an immunological age of an immune system refers to a profile, specifically a CD4 T cell profile typically obtained in subjects over the age of 70.

As used herein, the term “mature” when referring to an immunological age of an immune system refers to a profile, specifically a CD4 T cell profile typically obtained in subjects in the age of 40-70 or 50-70.

As used herein, the term “biomarker” refers to a nucleic acid sequence of a gene or a fragment thereof the expression of which is indicative of one or more subsets of CD4 T cells. The biomarker may be a serum biomarker released into circulation. Alternatively, the biomarker may be expressed at the cell surface of CD4 T cell. The biomarker may be DNA, mRNA or the cDNA corresponding thereto, which represent the gene or a fragment thereof. A polynucleotide may comprise modified nucleotides, such as methylated nucleotides and nucleotide analogs. The sequence of the biomarker may be interrupted by non-nucleotide components. A biomarker may be further modified after polymerization, such as by conjugation with a labeling component. The term also includes both double- and single-stranded molecules.

As used herein, the term “biomarker associated with the one or more subsets of CD4 T cells” may refers to any measurable indicator of the one or more subsets of CD4 T cells, such as expression levels (including single cell expression levels) of RNA and/or proteins associated with certain CD4 T cell phonotypes, such as but not limited to, CD4 cytotoxic cells and/or activated regulatory (aTreg) CD4 T cells. According to some embodiments, the markers (or some thereof) may be indicative of the subset of CD4 T-cells regardless of the activation status (whether activated or not). Alternatively, the markers (or some thereof) may be indicative of the subset of CD4 T-cells and their activation status (e.g. activated CD4 cytotoxic cell).

As used herein, the term “biomarker identifier” may refer to any molecule capable of identifying a biomarker. Non-limiting examples of biomarker identifiers include, RNA/DNA probes, primers, antibodies etc.

As used herein the term “CD4 cytotoxic cell” refers to a subset of CD4+ T cells with cytotoxic activity (CD4 CTL). These cells are characterized by their ability to secrete granzyme B and perforin and to kill the target cells in an MHC class II-restricted fashion.

As used herein the term “regulatory CD4 T cells” or “Treg” refer to a subpopulation of CD4+ T cells that modulate the immune system, maintain tolerance to self-antigens, and prevent autoimmune disease. As used herein the term “activated regulatory CD4 T cells” or “aTreg” refer to a subpopulation of Treg cells with an activated phenotype and a very strong inhibitory function on T cell proliferation.

As used herein, the term “exhausted CD4 T cells” refer to a subpopulation of CD4+ T cells characterized by poor effector functions and high expression of multiple inhibitory receptors.

As used herein the term “effector-memory T cells” or “TEM” refer to a subpopulation of antigen-experienced and long-surviving cells CD4+ T cells characterized by distinct homing capacity and effector function.

As used herein, the term “biological sample” may refer a sample obtained from a subject which is a body fluid or excretion sample including, but not limited to, seminal plasma, blood, peripheral blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, peritoneal fluid, cyst fluid, lavage of body cavities, broncho alveolar lavage, lavage of the reproductive system and/or lavage of any other organ of the body or system in the body and stool. Each possibility is a separate embodiment of the present invention.

In some embodiments, the biological sample, also termed hereinafter ‘the sample’, obtained from the subject comprises blood. In some embodiments, the sample obtained from the subject is peripheral blood. In some embodiments, the sample obtained from the subject comprises serum. In some embodiments, the sample obtained from the subject is a sample of serum.

In some embodiments, the term “peripheral blood”, as used herein, refers to blood comprising of red blood cells, white blood cells and platelets. Typically, the sample is a pool of circulating blood. According to some embodiments, the sample is a peripheral blood sample not sequestered within the lymphatic system, spleen, liver, or bone marrow.

In some embodiments, the sample is a plasma sample. In some embodiments, the sample is a plasma sample derived from peripheral blood.

As used herein, the term “isolate” of a biological sample refer to a subset, derivative or extract derived from the sample. A non-limiting example of an isolate of a biological sample are white blood cells derived from a blood sample. Another non-limiting example includes a T cell population or a CD4 T cell population derived from a blood sample.

As used herein the term “functionality” when referring to CD4 T-cells, e.g. CD4 cytotoxic T-cells or Treg cells refers to the “behavior” of the cells after their activation. According to some embodiments, the functionality of the CD4 T-cells may refer to the profile and/or level of cytokines and/or chemokines secreted by the cells (e.g. anti-CD3/anti-CD28, PMA, ConA). According to some embodiments, the profile and/or level of cytokines and/or chemokines secreted may provide an additional layer of validation regarding the status of the immune system (e.g. that cells are dysregulated).

In some embodiments, there is provided a method for evaluating an immunological age of an immune system of a subject, the method comprising:
obtaining a biological sample from a subject, the biological sample comprising biomarkers associated with one or more subsets of CD4 T cells;
detecting a level of a plurality of the biomarkers, thereby identifying the one or more subsets of CD4 T cells in the immune system, wherein the one or more subsets of CD4 T cells comprise at least one of cytotoxic T cells and activated regulatory (aTreg) CD4 T cells; and
evaluating the immunological age of the immune system based on the presence of the plurality of biomarkers identified in the biological sample.

In some embodiments, identifying the one or more subsets of CD4 T cells comprises determining the relative amount of each identified subset from the one or more subsets of CD4 T cells, compared to control value.

The term “control value” as used herein refers to a standard or reference value which represents the average, standard or normal number of CD4 T cells. This value can be a single value obtained from a single measurement or a mean value obtain from multiple measurements and/or multiple CD4 cell populations and/or CD4 cell populations derived from multiple biological samples. In some embodiments, the control value is a mean value obtained from a plurality of biological sample derived from human subjects. In some embodiments, the control value is an age-matched control. In some embodiments, the terms ‘control value’ and ‘age-matched control’ are exchangeable. In some embodiments, the control value comprises young threshold value, also termed hereinafter regulated, efficient and/or naïve threshold value and old threshold value also termed hereinafter dysregulated, aged, mature and/or exhausted threshold value, the former is calculated from a plurality of biological sample derived from young human subjects and the latter is calculated from a plurality of biological sample derived from old human subjects.

In some embodiments, the term “a plurality”, as used herein, refers to at least two. According to some embodiments, the term “a plurality” refers to at least 2, 3, 4, 5, 6, 7, 8, 9, 10, 11, 12, 13, 14, 15, 16 and 17.

In some embodiments, the term “a plurality of biomarkers”, as used herein, refers to at least two distinct biomarkers, each associated with a subset of CD4 T cells. In some embodiments, each distinct biomarker is associated with a distinct subset of CD 4 T cells. In some embodiments, the plurality of distinct biomarkers is associated with the same CD4 T cell subset.\

The term “comprising biomarkers associated with one or more subsets of CD4 T cells” refers to comprising a plurality of biomarkers associated with one or more subsets of CD4 T cells.

In some embodiments, young human subjects refer to human subjects who are at most 55 y.o. In some embodiments, young human subjects refer to human subjects who are at most 50 y.o. In some embodiments, young human subjects refer to human subjects who are at most 45 y.o. In some embodiments, young human subjects refer to human subjects who are at most 40 y.o. In some embodiments, young human subjects refer to human subjects who are at most 35 y.o. In some embodiments, young human subjects refer to human subjects who are at most 30 y.o.

In some embodiments, old human subjects refer to human subjects who are at least 45 y.o. In some embodiments, old human subjects refer to human subjects who are at least 50 y.o. In some embodiments, old human subjects refer to human subjects who are at least 55 y.o. In some embodiments, old human subjects refer to human subjects who are at least 60 y.o. In some embodiments, old human subjects refer to human subjects who are at least 65 y.o.

In some embodiments, the control value is relative number compared to a control value or compared to the total number of CD4 T cells in the biological sample.

The term “biological sample” as used herein refers to a sample obtained a subject, e.g. a human subject. The biological sample may be derived from biological fluids or tissues. Ise, the biological sample is derived from blood, serum, plasma, cerebrospinal fluid, urine, saliva, sputum, and pleural effusions. In addition, one of skill in the art would realize that certain biological samples would be more readily analyzed following a fractionation or purification procedure, e.g., separation of whole blood into serum and plasma components.

In some embodiments, the biological sample is an isolate derived from the biological sample. In some embodiments, the biological sample is a blood sample comprising circulating biomarkers associated with subsets of CD4 T cells. In some embodiments, the biological sample is a serum sample comprising circulating biomarkers associated with subsets of CD4 T cells. In some embodiments, the biological sample comprises one or more subsets of CD 4 T cells. In some embodiments, the biological sample is a blood sample comprising one or more subsets of CD 4 T cells. In some embodiments, the biological sample is a serum sample comprising one or more subsets of CD 4 T cells.

In some embodiments, the one or more subsets of CD4 T cells further comprise at least one of naïve CD4 T cells, Naïve_Isg15 CD4 T cells, effector memory (TEM) CD4 T cells, exhausted CD4 T cell and, rTregs CD4 T cells.

In some embodiments, determining the expression levels of the biomarkers may comprise detection of the expression or expression levels of specific nucleic acid sequences via any means known in the art, and as described herein.

In some embodiments, evaluating the immunological age of the immune system comprises evaluating the presence of each subset in the biological sample based on the level of the plurality of biomarkers associated therewith, comparing the level of the plurality of biomarkers to mature threshold value, wherein detecting a high probability score for said level with respect to the mature threshold value indicates that the immunological age of the immune system is mature.

In some embodiments, “detecting a level of a plurality of biomarker” comprises assessing the presence, absence, quantity or relative amount (which can be an “effective amount”) of each biomarker in the plurality of biomarkers, within a clinical or subject-derived sample, including qualitative or quantitative concentration levels of such biomarker.

In some embodiments, “detecting a level of a plurality of biomarker” comprises determining the expression level of each biomarker of said plurality of biomarkers or determining the amount, or relative amount, of DNA or cDNA corresponding to the expression level of mRNA biomarker(s).

In some embodiments, evaluating the immunological age of the immune system comprises evaluating the presence of each subset in the biological sample based on the level of the plurality of biomarkers associated therewith, comparing the level of the plurality of biomarkers to young threshold value, wherein detecting a high probability score for said level with respect to the young threshold value indicates that the immunological age of the immune system is young.

In some embodiments, detecting a high probability score with respect to a given threshold value, for example, mature threshold value, comprises comparing the level of the plurality of biomarkers in the sample to a corresponding reference/threshold value, and assigning a probability score reflecting the probability that the said level is mature (i.e. representing mature immunological age) based on the comparison, wherein a high probability score, above a predefined threshold, is indicative of mature immunological age.

In some embodiments, the control value is a reference scale comprising a plurality of threshold values comprising a value or a range of values representing each CD4 T cell subset in young and in mature immune system. Thus, evaluating the immunological age of the immune system comprises evaluating the presence of each subset in the biological sample based on the level of the plurality of biomarkers associated therewith, wherein said probability score is a score assigned to said level of the plurality of biomarkers based on its relative position within the reference scale.

In some embodiments, the one or more subsets of CD4 T cells further comprises exhausted CD4 T cells. In some embodiments, the one or more subsets of CD4 T cells further comprises TEM CD4 T cells. In some embodiments, the one or more subsets of CD4 T cells further comprises rTregs CD4 T cells. In some embodiments, the one or more subsets of CD4 T cells further comprises naïve CD4 T cells. In some embodiments, the one or more subsets of CD4 T cells further comprises naïve_Isg15 CD4 T cells CD4 T cells.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells above 50%, indicates that the immunological age of the immune system is young.

It is to be understood that a value above or below a certain threshold, such as, above 50%, may be obtained by any method known in the art, for example, by comparing the level of the plurality of biomarkers associated with a certain subset of CD4 T cells, to the level of all biomarkers measured in the biological sample.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells above 55%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells above 60%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells below 50%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells below 45%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells below 40%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 above 50%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 above 55%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 above 60%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 below 50%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 below 45%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with naïve CD4 T cells and naïve_Isg15 below 40%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells above 1%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells above 2%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells above 5%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells below 1%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells below 0.5%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with cytotoxic CD4 T cells below 0.2%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells above 1%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells above 3%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells above 5%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells above 8%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells below 1%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells below 0.8%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with exhausted CD4 T cells below 0.5%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with aTregs CD4 T cells above 1%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with aTregs CD4 T cells above 3%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with aTregs CD4 T cells above 5%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with aTregs CD4 T cells below 3%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with aTregs CD4 T cells below 1%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with TEM CD4 T cells above 4%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with TEM CD4 T cells above 5%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with TEM CD4 T cells above 10%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with TEM CD4 T cells below 4%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with TEM CD4 T cells below 3%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells above 9%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells above 15%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells above 20%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells above 25%, indicates that the immunological age of the immune system is mature.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells below 9%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells below 5%, indicates that the immunological age of the immune system is young.

In some embodiments, evaluating the immunological age of the immune system comprises evaluating the presence of each subset in the biological sample based on the level of the plurality of biomarkers associated therewith, wherein a level of the plurality of biomarkers associated with two or more CD4 T cells subsets selected from TEM CD4 T cells, cytotoxic CD4 T cells, exhausted CD4 T cells and aTregs CD4 T cells below 3%, indicates that the immunological age of the immune system is young.

In some embodiments, the level of each biomarkers may be measured electrophoretically or immunochemically, wherein the immunochemical detection may be achieved by radioimmunoassay, immunofluorescence assay or by an enzyme-linked immunosorbent assay. In some embodiments, the level of each biomarkers is measured by qPCR.

In some embodiments, evaluating the immunological age of the immune system comprises evaluating the relative amount of each subset in the biological sample,

In some embodiments, the immunological age of an immune system is identified as dysregulated/abnormal when the relative amount of aTreg CD4 T cells, CD4 cytotoxic T cells and exhausted CD4 T cells deviates from regulated immune system control.

The term “regulated immune system control” as used herein refers to a control/threshold value corresponding to a range of values corresponding to regulated immune system, wherein values deviating from said regulated immune system control indicate that the corresponding immune system is dysregulated.

It is to be understood that deviation from regulated immune system control means that the level of aTreg CD4 T cells, CD4 cytotoxic T cells and exhausted CD4 T cells is significantly lower or significantly higher from the value/values of immune system control. Stated otherwise, deviation from regulated immune system control means that the level of aTreg CD4 T cells, CD4 cytotoxic T cells and exhausted CD4 T cells is having a low probability score when compared to value/values of immune system control.

In some embodiments, evaluating the immune system as a dysregulated immune system comprises evaluating the presence of aTreg CD4 T cells, CD4 cytotoxic T cells and exhausted CD4 T cells in the biological sample based on the level of at least one biomarker associated with immune system dysregulation, comparing the level of the at least one biomarker to regulated immune system control, wherein detecting a low probability score for said level of the at least one biomarker associated with immune system dysregulation with respect to the regulated immune system control indicates that the immune system is dysregulated.

In some embodiments, evaluating the immunological age of the immune system comprises determining the relative amount of aTreg CD4 T cells, CD4 cytotoxic T cells and exhausted CD4 T cells, based on the level of a plurality of biomarkers associated with aTreg CD4 T cells, CD4 cytotoxic T cells and exhausted CD4 T cells.

The term “relative amount” with respect to one or more subsets of CD4 T cells in a sample refers to the amount of the one or more subsets relative to the total amount of CD4 T cells, in the sample.

In some embodiments, the immunological age of an immune system is identified as mature when the relative amount of aTreg CD4 T cells, CD4 cytotoxic T cells and exhausted CD4 T cells exceeds 15%.

In some embodiments, the plurality of biomarkers are selected from the group consisting of: EOMES, CCL3, CCL4, CCL5, CCR7, CD7, CD8, CD74, CD137, CD134, CD25, CD44, CD62L, CD81, CD200, Cst7, Ms4a4b, NKG2D, Nfatc1, Runx2, Runx3, Tbx21, GzmB, GzmK, perforin, FOXP3, GITR, Helios, Lgals1, IGFbp4, LAG3, IL-1a, IL-1b, IL1R2, IL2RA, IL-6, IL-10, IL-17A, IL-21, IL-18R1, IL-27, IFN-b, IFN-g, Isg15, PD1, Left, Lfit3, MCP1, Satbl, Ccr7, Aw112010, S100a10, S100a11, S100a4, Sell, Pdcd1, Izumo1r, Ikzf2, Igfbp4, Itgb1, Itgb7, GM-CSF, Sostdc1, Tbc1d4, TNF, TNFRSF4, TNFSF8, TNFRSF8, Ctla2a, Ctla4 and TNF-RSF9/4.

In some embodiments, the plurality of biomarkers comprises at least two biomarkers. In some embodiments, the plurality of biomarkers comprises at least three biomarkers. In some embodiments, the plurality of biomarkers comprises at least four biomarkers.

In some embodiments, at least one biomarker from the plurality of biomarkers is EOMES. In some embodiments, at least one biomarker from the plurality of biomarkers is CCL3. In some embodiments, at least one biomarker from the plurality of biomarkers is CCL4. In some embodiments, at least one biomarker from the plurality of biomarkers is CCL5. In some embodiments, at least one biomarker from the plurality of biomarkers is CCR7. In some embodiments, at least one biomarker from the plurality of biomarkers is CD7. In some embodiments, at least one biomarker from the plurality of biomarkers is CD8. In some embodiments, at least one biomarker from the plurality of biomarkers is CD137. In some embodiments, at least one biomarker from the plurality of biomarkers is CD134. In some embodiments, at least one biomarker from the plurality of biomarkers is CD25. In some embodiments, at least one biomarker from the plurality of biomarkers is CD44. In some embodiments, at least one biomarker from the plurality of biomarkers is CD62L. In some embodiments, at least one biomarker from the plurality of biomarkers is CD74. In some embodiments, at least one biomarker from the plurality of biomarkers is CD81. In some embodiments, at least one biomarker from the plurality of biomarkers is CD200. In some embodiments, at least one biomarker from the plurality of biomarkers is Cst7. In some embodiments, at least one biomarker from the plurality of biomarkers is Ms4a4b. In some embodiments, at least one biomarker from the plurality of biomarkers is NKG2D. In some embodiments, at least one biomarker from the plurality of biomarkers is Nfatc1. In some embodiments, at least one biomarker from the plurality of biomarkers is Runx2. In some embodiments, at least one biomarker from the plurality of biomarkers is Runx3. In some embodiments, at least one biomarker from the plurality of biomarkers is Tbx21. In some embodiments, at least one biomarker from the plurality of biomarkers is GzmB In some embodiments, at least one biomarker from the plurality of biomarkers is GzmK In some embodiments, at least one biomarker from the plurality of biomarkers is perform. In some embodiments, at least one biomarker from the plurality of biomarkers is FOXP3. In some embodiments, at least one biomarker from the plurality of biomarkers is GITR. In some embodiments, at least one biomarker from the plurality of biomarkers is Helios. In some embodiments, at least one biomarker from the plurality of biomarkers is Lgals 1. In some embodiments, at least one biomarker from the plurality of biomarkers is IGFbp4. In some embodiments, at least one biomarker from the plurality of biomarkers is LAG3. In some embodiments, at least one biomarker from the plurality of biomarkers is Isg15. In some embodiments, at least one biomarker from the plurality of biomarkers is IL-1a. In some embodiments, at least one biomarker from the plurality of biomarkers is IL-1b. In some embodiments, at least one biomarker from the plurality of biomarkers is IL1R2. In some embodiments, at least one biomarker from the plurality of biomarkers is IL2RA. In some embodiments, at least one biomarker from the plurality of biomarkers is IL-6. In some embodiments, at least one biomarker from the plurality of biomarkers is IL-10. In some embodiments, at least one biomarker from the plurality of biomarkers is IL-17A. In some embodiments, at least one biomarker from the plurality of biomarkers is IL-21. In some embodiments, at least one biomarker from the plurality of biomarkers is IL-18R1. In some embodiments, at least one biomarker from the plurality of biomarkers is IL-27. In some embodiments, at least one biomarker from the plurality of biomarkers is IFN-b. In some embodiments, at least one biomarker from the plurality of biomarkers is IFN-g. In some embodiments, at least one biomarker from the plurality of biomarkers is GM-CSF. In some embodiments, at least one biomarker from the plurality of biomarkers is PD1. In some embodiments, at least one biomarker from the plurality of biomarkers is Left. In some embodiments, at least one biomarker from the plurality of biomarkers is Lfit3. In some embodiments, at least one biomarker from the plurality of biomarkers is MCP1. In some embodiments, at least one biomarker from the plurality of biomarkers is Satb1. In some embodiments, at least one biomarker from the plurality of biomarkers is Ccr7. In some embodiments, at least one biomarker from the plurality of biomarkers is Aw112010. In some embodiments, at least one biomarker from the plurality of biomarkers is S100a1 1. In some embodiments, at least one biomarker from the plurality of biomarkers is S100a10. In some embodiments, at least one biomarker from the plurality of biomarkers is S100a4. In some embodiments, at least one biomarker from the plurality of biomarkers is Sell. In some embodiments, at least one biomarker from the plurality of biomarkers is Sostdc1. In some embodiments, at least one biomarker from the plurality of biomarkers is Pdcd1. In some embodiments, at least one biomarker from the plurality of biomarkers is Izum1r. In some embodiments, at least one biomarker from the plurality of biomarkers is Igfbp4. In some embodiments, at least one biomarker from the plurality of biomarkers is Ikzf2. In some embodiments, at least one biomarker from the plurality of biomarkers is Itgb 1. In some embodiments, at least one biomarker from the plurality of biomarkers is Tbcld4. In some embodiments, at least one biomarker from the plurality of biomarkers is TNF. In some embodiments, at least one biomarker from the plurality of biomarkers is TNFRSF4. In some embodiments, at least one biomarker from the plurality of biomarkers is TNFSF8. In some embodiments, at least one biomarker from the plurality of biomarkers is TNFRSF8. In some embodiments, at least one biomarker from the plurality of biomarkers is Ctla2a. In some embodiments, at least one biomarker from the plurality of biomarkers is Ctla4. In some embodiments, at least one biomarker from the plurality of biomarkers is TNF-RSF9/4.

In some embodiments, the plurality of biomarkers is selected from EOMES+, CCL5+, NKG2D+, Perforin, RunX3, NKg7 and GzmB, the one or more subsets of CD4 T cells identified in the biological sample is CD4 cytotoxic cell. Each possibility is a separate embodiment of the present invention.

In some embodiments, the association between a plurality of biomarkers and a subset of cells or an immunological age is determined by the presence of a specific biomarker or a combination of biomarkers. Alternatively, the association between a plurality of biomarkers and a subset of cells or an immunological age is determined by the level of the plurality of biomarkers relative to a predetermined threshold.

In some embodiments, the plurality of biomarkers is selected from CD137, CD134, FOXP3+, GITR+, Helios+, CD74, HLA-DR, wherein the one or more subsets of CD4 T cells identified in the biological sample is aTreg CD4 T cell. Each possibility is a separate embodiment of the present invention.

In some embodiments, the plurality of biomarkers is selected from Nkg7, Runx3, Eomes, IFN-b, IL-27 and Gzmk wherein the one or more subsets of CD4 T cells identified in the biological sample is CD4 cytotoxic cells. Each possibility is a separate embodiment of the present invention.

In some embodiments, the plurality of biomarkers is selected from Nkg7, Runx3, Eomes, Gzmk, IFN-b, IL-27, IL21, IL 17A, Cc13, Cc14 and Cc15, wherein the one or more subsets of CD4 T cells identified in the biological sample is CD4 cytotoxic cells. Each possibility is a separate embodiment of the present invention.

In some embodiments, the plurality of biomarkers is selected from Cd200, Lag3, Hif1a, Nfatc1 and Pdcd1, wherein the one or more subsets of CD4 T cells identified in the biological sample is exhausted CD4 T cells. Each possibility is a separate embodiment of the present invention.

In some embodiments, the plurality of biomarkers comprises at least one of Itgb7 and IL-18R1, wherein the one or more subsets of CD4 T cells identified in the biological sample is TEM CD4 T cells.

In some embodiments, the plurality of biomarkers comprises at least one of Ms4a4b and Sell, wherein the one or more subsets of CD4 T cells identified in the biological sample is rTregs CD4 T cells.

In some embodiments, the plurality of biomarkers is selected from Pdcd1, Lag3, TNFRSF4, S100a11, IL1R2 and Ikzf2, wherein the one or more subsets of CD4 T cells identified in the biological sample is aTregs CD4 T cells. Each possibility is a separate embodiment of the present invention.

In some embodiments, the plurality of biomarkers comprises PD1, TNFRSF4, TNFRSF9, Helios, IL1R2 and LAG3 and the immunological age of the immune system is mature. Each possibility is a separate embodiment of the present invention.

In some embodiments, the plurality of biomarkers is selected from PD1, TNFRSF4, TNFRSF9, Helios, IL1R2 and LAG3 and the immunological age of the immune system is mature. Each possibility is a separate embodiment of the present invention.

In some embodiments, the plurality of biomarkers is selected from CCR7, CD44, LAG3, PD1, FOXP3 and CD81 and the immunological age of the immune system is mature. Each possibility is a separate embodiment of the present invention.

In some embodiments, the plurality of biomarkers is selected from Nkg7, Runx3, Eomes, IFN-b, IL-27 and Gzmk and the immunological age of the immune system is mature. Each possibility is a separate embodiment of the present invention.

In some embodiments, the plurality of biomarkers is selected from Nkg7, Runx3, Eomes, Gzmk, IFN-b, IL-27, IL21, IL 17A, Cc13, Cc14 and Cc15 and the immunological age of the immune system is mature. Each possibility is a separate embodiment of the present invention.

In some embodiments, the plurality of biomarkers comprises at least two of Left, Satb1, Ccr7, Aw112010, S100a11, IL-27 and Izumo lr and the immunological age of the immune system is mature. Each possibility is a separate embodiment of the present invention. In some embodiments, the plurality of biomarkers comprises Lef1, Satb1 and Ccr7 and the immunological age of the immune system is mature. Each possibility is a separate embodiment of the present invention. In some embodiments, the plurality of biomarkers comprises Aw112010 and S100a11 and the immunological age of the immune system is mature. In some embodiments, the plurality of biomarkers comprises IL-27 and the immunological age of the immune system is mature.

In some embodiments, when the level of CD4 cytotoxic cells exceeds the value of regulated immune system control the immune system is identified as dysregulated.

In some embodiments, when the level of CD4 cytotoxic cells exceeds the value of regulated immune system control the immune system is identified as mature.

In some embodiments, when the level of aTreg CD4 cells exceeds the value of regulated immune system control the immune system is identified as mature.

In some embodiments, when the level of aTreg CD4 cells exceeds the value of regulated immune system control the immune system is identified dysregulated.

According to some embodiments, obtaining a biological sample comprising tissue or fluid is carried out by any one or more of the following collection methods blood sampling, urine sampling, stool sampling, sputum sampling, aspiration of pleural or peritoneal fluids, fine needle biopsy, needle biopsy, core needle biopsy and surgical biopsy, and lavage. Each possibility is a separate embodiment of the present invention. Regardless of the procedure employed, once a biopsy/sample is obtained the level of the plurality of biomarkers can be determined and evaluation can thus be made.

In some embodiments, the method further comprising isolating a CD4 T cell population from the biological sample.

In some embodiments, the method further comprises the step of treating the subject with a therapeutically effective agent for the treatment of age-related frailty.

Frailty is a clinical syndrome with symptoms as low body weight due to unintentional weight loss, exhaustion, weakness, slow walking and low physical activity. It is typically characterized by decreased reserves and reduced resistance to stressors, causing a vulnerability to adverse outcomes. In addition, frailty is associated with increased insulin resistance, metabolic syndrome and osteoporosis. Frailty was shown to be associated with changes in biomarkers, such as, IL-6, CRP, 25-OH-vitamin-D, IGF-1 and D-dimers. In some embodiments, frailty is prominent in elderly people.

Therapies for the treatment of frailty in the elderly, include nutritional supplementation for example, vitamins, carotenoids, creatine, DHEA, and/or beta-hydroxy-beta-methylbutyrate. Supplementation with vitamin D is known to alleviate syndromes of frailty.

In this context, the combination of anabolic steroid, such as, nandrolone decanoate, alphacalcidol (Vitamin D3) and calcium was shown to improve bone mineral density, muscle mass and gait scores in women recovering from hip fraction.

In some embodiments, the age-related frailty is an immune-associated disease.

In some embodiments, the method further comprises the step of treating the subject with a therapeutically effective agent for the treatment of a disease or disorder. In some embodiments, the disease or disorder is an age-related disease or disorder.

In some embodiments, the age-related of disease or disorder is selected form the group of Alzheimer's disease, depression, short-term memory loss, urinary incontinence, osteoporosis, osteoarthritis, Parkinson's disease, malignant diseases (various types of cancer), hypertension, cardiovascular disease, amyotrophic lateral sclerosis (ALS), heart diseases, diabetes, inflammation (such as, systemic inflammation), dementia, neurodegenerative diseases, cellular immune dysfunction and obesity. Each possibility is a separate embodiment of the present invention.

It is acknowledged by the healthcare community that an individuals' response to disease and their ability to metabolize medicines changes with age. The changing world demographics due to increasing life expectancy, with increasing numbers of older people (also called ‘silver tsunami’), raises the need to develop assays and medicines designed for the specific needs that are prevalent in the older population. Thus, the method disclosed herein addresses the aforementioned need by providing a platform for assessing the immunological age of an immune system and accordingly designing a treatment suitable for the immunological age of a subject, which can reduce side effects and avoid administration of ineffective treatments.

In some embodiments, the age-related disease or disorder is a proliferative disease or disorder. In some embodiments, the age-related disease or disorder is cancer.

In some embodiments, the age-related disease or disorder is an infectious disease. In some embodiments, the age-related disease or disorder is associated with immunodeficiency disorder. In some embodiments, the age-related disease or disorder is chronic inflammation

In some embodiments, the agent for the treatment of the age-related disease or disorder is selected from statins, antibiotics, chemotherapy, steroids, nonsteroidal anti-inflammatory drug or a combination thereof.

In some embodiments, the agent is a vaccine and the treatment is adaptive immunity to a specific disease or disorder, wherein the choice of vaccine or the amount of vaccine depends on the immunological age of the immune system of the subject.

In some embodiments, the agent is a live attenuated vaccine.

In some embodiments, the agent is a substance capable of targeting at least one of the biomarkers in the plurality of biomarker. In some embodiments, the agent is selected from the group consisting of an antibody, a siRNA, a microRNA, a small molecule or any combination thereof.

In some embodiments, the antibody is an NKG2D antibody, a CD7 antibody, a CD134 antibody, a CD137 antibody, a GITR antibody or any combination thereof.

In some embodiments, there is provided a therapeutically effective agent for the treatment of an age-related disease or disorder. In some embodiments, the age-related disease or disorder is a proliferative disease or disorder. In some embodiments, the age-related disease or disorder is cancer. In some embodiments, the age-related disease or disorder is an infectious disease. In some embodiments, the age-related disease or disorder is associated with immunodeficiency disorder. In some embodiments, the age-related disease or disorder is chronic inflammation

In some embodiments, the therapeutically effective agent for the treatment of the age-related disease or disorder is selected from statins, antibiotics, chemotherapy, steroids, nonsteroidal anti-inflammatory drug or a combination thereof.

In some embodiments, the therapeutically effective agent is a vaccine and the treatment is adaptive immunity to a specific disease or disorder, wherein the choice of vaccine or the amount of vaccine depends on the immunological age of the immune system of the subject.

In some embodiments, the therapeutically effective agent is a live attenuated vaccine.

In some embodiments, the therapeutically effective agent is a substance capable of targeting at least one of the biomarkers in the plurality of biomarker. In some embodiments, the agent is selected from the group consisting of an antibody, a siRNA, a microRNA, a small molecule or any combination thereof. In some embodiments, the antibody is an NKG2D antibody, a CD7 antibody, a CD134 antibody, a CD137 antibody, a GITR antibody or any combination thereof.

In some embodiments, there is provided a method of treating age-related frailty, the method comprising the steps of:
obtaining a biological sample from a subject, the biological sample comprising biomarkers associated with one or more subsets of CD4 T cells;
detecting a level of a plurality of the biomarkers, thereby identifying the one or more subsets of CD4 T cells in the immune system, wherein the one or more subsets of CD4 T cells comprise at least one of cytotoxic T cells and activated regulatory (aTreg) CD4 T cells;
determining the immunological age of the immune system as mature based on the presence of the plurality of biomarkers identified in the biological sample; and
treating the subject with a therapeutically effective agent for the treatment of age-related frailty.

In some embodiments, there is provided a pharmaceutical composition for the treatment of age-related frailty, wherein the age-related frailty is determined based on the presence of a plurality of biomarkers, identified in a biological sample, the plurality of the biomarkers is associated with one or more subsets of CD4 T cells in an immune system corresponding to the biological sample, wherein the plurality of biomarkers indicate the presence of subsets of CD4 T cells comprising at least one of cytotoxic T cells and activated regulatory (aTreg) CD4 T cells, and wherein said pharmaceutical composition comprises a therapeutically effective agent for the treatment of age-related frailty.

In some embodiments, the pharmaceutical composition may contain pharmaceutically acceptable concentrations of salt, buffering agents, preservatives and various compatible carriers. Each possibility is a separate embodiment. For all forms of delivery, the pharmaceutical composition may be formulated in a physiological salt solution. In some embodiments, the pharmaceutical composition may be incorporated in a liposome or other biomaterial useful for protecting and/or preserving the therapeutically effective agent, until it is delivered to the target cell or tissue. The liposome may also help target the therapeutically effective agent to a desired location, e.g., a tumor.

The pharmaceutical composition disclosed herein may be prepared by known methods for the preparation of pharmaceutically acceptable compositions suitable for administration to patients, such that an effective quantity of the therapeutically effective agent, and any additional active substance or substances, is combined in a mixture with a pharmaceutically acceptable vehicle. On this basis, the pharmaceutical composition recited herein may include, albeit not exclusively, solutions of the active ingredients in association with one or more pharmaceutically acceptable vehicles or diluents and may be contained in buffer solutions with a suitable pH and iso-osmotic with physiological fluids.

The proportion and identity of a pharmaceutically acceptable diluent used in the pharmaceutical composition may be determined by the chosen route of administration, compatibility with live cells, and standard pharmaceutical practice. Generally, a pharmaceutical composition is formulated with components that do not destroy or significantly impair the biological properties of the active ingredients.

In some embodiments, the pharmaceutical composition is administered locally, e.g., in a tumor, or systemically.

The term “treating” as used herein refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of spread or development of the disease or condition, delay or slowing of disease progression, amelioration or palliation of the disease state, and remission (whether partial or total). “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently.

In some embodiments, the subject in need thereof is a subject in need of treatment or prevention, who is human. The subject may be a subject who has an age-related frailty, such as a subject who has cancer, or a subject likely to develop cancer.

It is to be understood that evaluating the immunological age of the immune system based on the presence of the plurality of biomarkers identified in the biological sample as disclosed herein is a statistical evaluation, namely, the evaluation is obtained by various statistical means and hence refers to the tendency, or probability of the tested immune system (or biological sample corresponding thereto) to be mature, young or dysregulated.

In some embodiments, there is provided a kit for evaluating an immunological age of an immune system in a biological sample, the kit comprising: (a) means for measuring the level of a plurality of biomarkers associated with one or more subsets of CD4 T cells, in a biological sample; and (b) means for determining a threshold value for each biomarker in the plurality of biomarkers or information regarding the cutoff value, wherein a level of the plurality of biomarkers relative to said cutoff value identifies the immunological age of the immune system.

In some embodiments, the means for measuring the level of a plurality of biomarkers comprises one or more biomarker identifiers. Thus, the term ‘means for measuring the level of a plurality of biomarkers’ as used herein is exchangeable with the term a plurality of biomarker identifier, wherein each biomarker identifier in the plurality of biomarker identifiers is configured to determine a level of a biomarker associated with a subset of CD4 T cells.

In some embodiments, the means for measuring the levels of a biomarker may include a plurality of oligonucleotides, each capable of amplifying at least one biomarker in the plurality of biomarkers, include a plurality of oligonucleotides, each capable of hybridizing at least one biomarker in the plurality of biomarkers, a plurality of nucleotide primer pairs each adjusted to flank at least one biomarker in the plurality of biomarkers and a combination thereof.

As used herein, a “primer” defines an oligonucleotide which is capable of annealing to (hybridizing with) a target sequence, thereby creating a double stranded region which can serve as an initiation point for DNA synthesis under suitable conditions.

In some embodiments, the terminology “primer pair” refers herein to a pair of oligonucleotides (oligos), which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In some embodiments, oligonucleotide primers may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence.

In some embodiments, each of the oligonucleotides capable of hybridizing to the at least one biomarker in the plurality of biomarkers comprises a detectable label.

In some embodiments, the detectable label produces a signal that correlates with the level of said at least one biomarker. In some embodiments, the detectable label produces an optical signal.

In some embodiments, said means is a plurality of nucleotide primer pairs each adjusted to flank at least one biomarker in the plurality of biomarkers and each of the primers comprises a detectable label.

In some embodiments, the kit further comprising instructions of use thereof evaluating an immunological age of an immune system in a biological sample.

### EXAMPLES

### Materials and Methods

### Mice

WT C57BL/6 and CD45.1 (B6.SJL-Ptprca Pepcb/BoyJ) mice were purchased from the Jackson Laboratory (Bar Harbor, ME) and were housed under specific pathogen-free conditions at the animal facility of Ben-Gurion University. WT C57BL/6 Mice were kept in different age batches from 2 to 24 months. All mice were checked for any macroscopic abnormalities (according to the Jackson guide—“AGED C57BL/6J MICE FOR RESEARCH STUDIES”). Animals with skin lesions, organ specific problems, or behavioral issues were discarded from the study. All surgical and experimental procedures were approved by the Institutional Animal Care and Use committee (IACUC) of Ben-Gurion University of the Negev, Israel.

Sample Processing for Single-Cell RNA Sequencing (scRNA-seq)

Young (2-3 months) and old (22-24 months) mice were sacrificed using overdose of Isoflurane. Then, spleens were harvested and mashed into 70 pm cell strainer. Lysis of red blood cells was preformed using 300 μl ASK buffer for 1 minute (Lonza, Basel Switzerland). CD4⁺ T cells were then purified from total splenocytes using a magnetic microbeads negative selection kit (EasySep™ CD4⁺ T Cell Isolation Kit, StemCell Technologies), according to the manufacturer's instructions. In all processing steps, wide-bore pipette tips were used, and centrifugation condition did not exceed 400 RCF, to minimize physical damage to the cells caused by shearing forces.

FACS sorting: To enhance purity and discard dead cells, CD4 T cells were further purified using FACS. Cells were sorted as eFluor780-Live cells⁺ (Fixable Viability dye, eBioscience), PerCP/cy5.5-conjugated anti-CD8⁻ (53-6.7; BioLegend), PE-conjugated anti-CD19⁻ (6D5; BioLegend), PE-conjugated anti-CD11b⁻ (M170; BioLegend), PE-conjugated anti-NK1.1⁻ (PK136; BioLegend), BV421-conjugated anti-CD4⁺ (GK1.5; BioLegend) and FITC-conjugated anti-TCRb⁺ (H57-597; BioLegend) using the FACSAria™ III instrument (BD Biosciences, San Jose, Calif.). To minimize physical stress to the cells, 100 μm nozzle and 20 PSI sheet fluid pressure were used. Cells were sorted into RPMI (Life Technologies) enriched media with 10% FCS, 10 mM HEPES, 1 mM sodium pyruvate, 10 mM nonessential amino acids, 1% Pen/Strep/Nystatin and 50 μM 2-ME. After sorting, CD4 T cells in all samples reached >99% purity. Prior to the loading on Chromium Controller Instrument (10× Genomics), cells were suspended in PBS supplemented with 0.04% BSA, counted 4 times under a light microscope and passed through a 40 pm strainer (for extended protocol see “Cell preparation guide”, 10× Genomic protocol; https://support.10xgenomics.com/single-cell-gene-expression/sample-prep). To reach 2000-3000 recovered cells, ˜5000 cells per sample were loaded to 10× Genomics platform.

### Tissue Processing for Flow Cytometry and In Vitro Assays

Spleen: see Materials and methods, section “Samples processing for single-cell RNA sequencing (scRNA-seq)”.

Lymph nodes: Mice were killed with overdose of isoflurane and lymph nodes were harvested from inguinal, mesenteric, cervical and axillar areas. Then, lymph nodes were mashed into 70pm cell strainer and cells were washed and counted.

Blood: Blood was collected into EDTA-coated tubes (MiniCollect, Greiner Bio-One) from euthanized mice using cardiac puncture. Red blood cells were then lysed using blood lysis buffer (BD bioscience) and the remaining leukocytes were washed twice and counted.

Bone marrow: Mice were killed with overdose of isoflurane. Femurs and tibias were collected. Cells from the bone marrow were obtained by flushing the bones with injected sterile PBS. Red blood cells were removed using 500 pl ACK lysis buffer for 1.5 minutes.

Sequencing Library Construction using the 10× Genomics Chromium Platform

Single-cell suspension was loaded on a Chromium Controller Instrument (10× Genomics) to generate single-cell gel bead-in-emulsions (GEMs). Barcoding and cDNA libraries were prepared using the Single-Cell 3′ Library & Gel Bead kit, according to manufacturer instructions. GEM-reverse transcription (GEM-RT) was performed in a Mastercycler nexus Thermal cycler (Eppendorf). After RT, GEMs were broken, and the cDNA was cleaned up with DynaBeads MyOne Silane Beads (Thermo Fisher Scientific) and amplified Amplified cDNA product was cleaned up with the SPRIselect Reagent Kit (0.6× SPRI; Beckman Coulter). Indexed sequencing libraries were constructed using the Chromium Single-Cell 3′ Library Kit (10x Genomics) for enzymatic fragmentation, end-repair, A-tailing, adaptor ligation, post-ligation cleanup, sample index PCR and PCR cleanup. Final libraries were quantified by Qubit, Tape station and quantitative PCR using NEBNext Library Quant Kit (BioLabs). Libraries were loaded at 2-2.2 pM on an Illumina NextSeq500 using the high output 75-cycles kit with the following parameters: 26 bp for Readl, 56 bp for Read2 and 8 bp for 17 Index. Each library was sequenced twice using the Illumina's NextSeq 500 sequencing platform. Sequencing depth was 50,000-100,000 reads per cell.

### RNA Sequencing Data Processing

The output Illumina base call files (BCLs) from both sequencing runs were merged and converted to FASTQ files using Cell Ranger software v2.0.1, which included Illumina's bcl2fastq v2.19.1.403. FASTQ files of each mouse sample were converted to count matrices using Cell Ranger software, based on transcriptome reference of Mus Musculus (mm10 1.2.0). 25,707 barcodes that were identified as cells, according to Cell Ranger's pipeline of calling cell barcodes, were used for further analysis.

### Quality Control (QC)

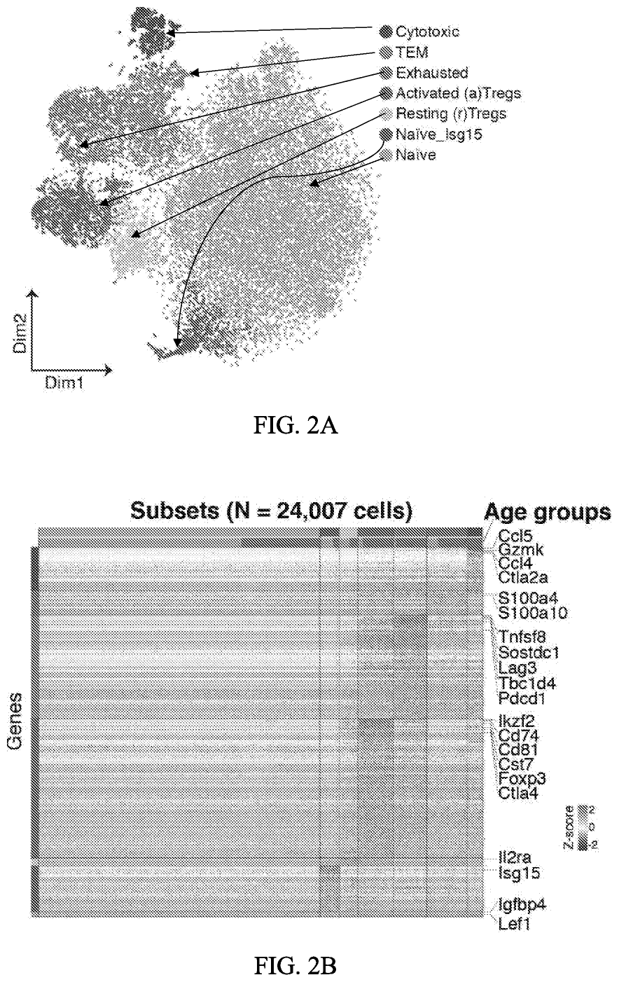

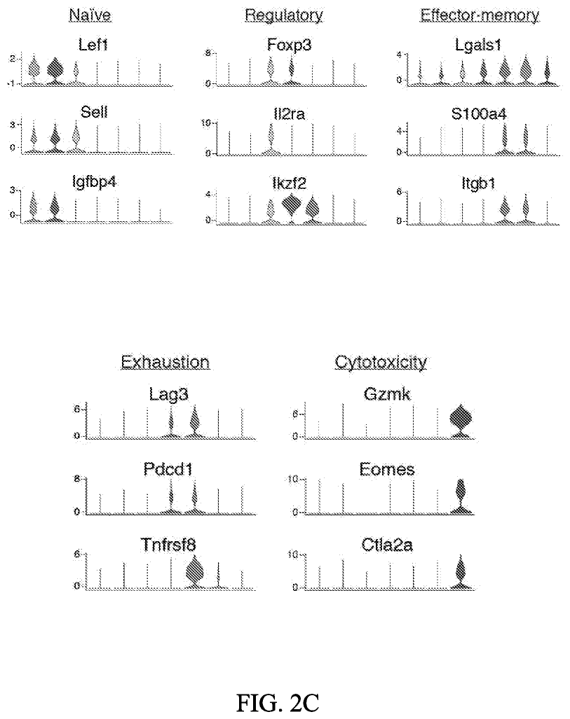

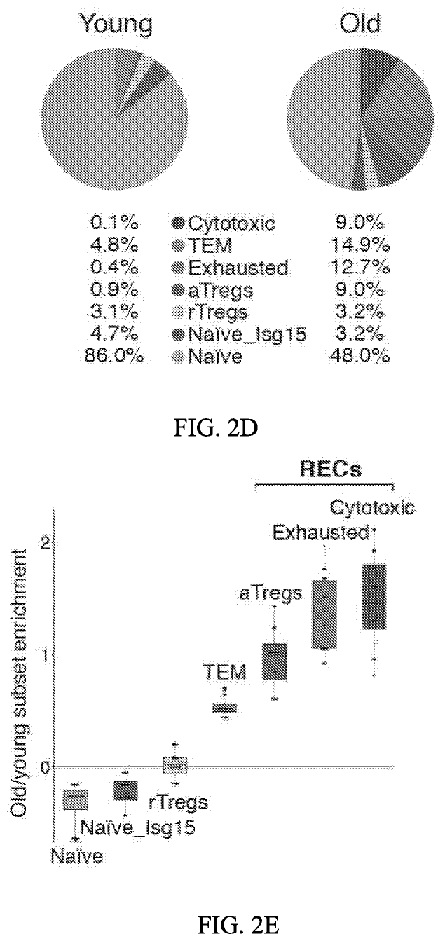

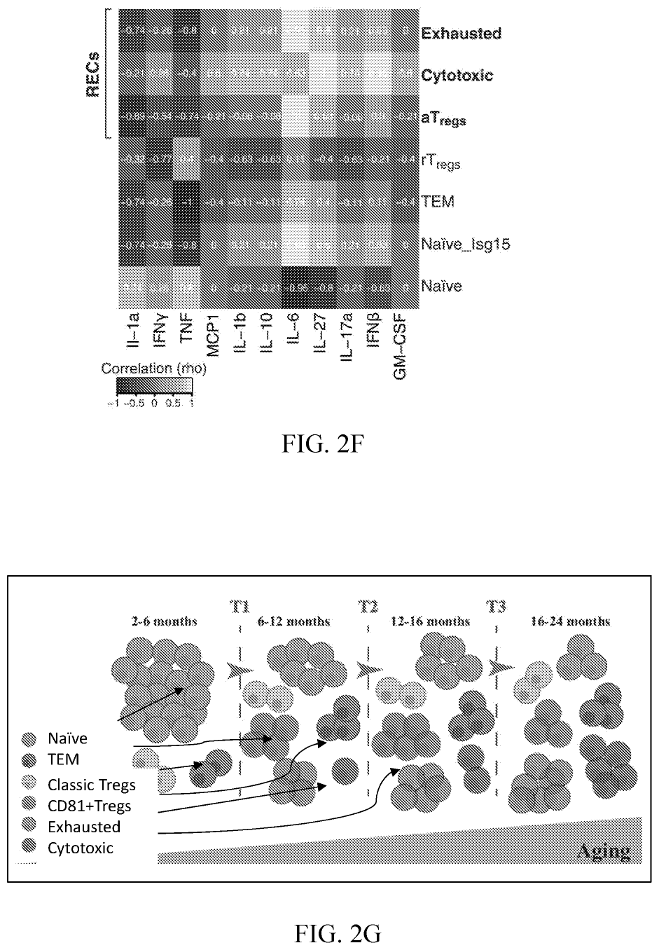

QC and further analyses were done in R v3.4.2, using Seurat package v2.0.1 (22); Supplementary FIG. 2A). To discard doublets, cells were ordered by their number of unique molecular identifiers (UMIs), and the top (N/1,000) % of cells per sample were filtered out, where N is the total number of called cell barcodes identified per sample (18). To discard broken cells (lysed cells with retained mitochondrial RNA), cells with more than 5% UMIs mapped to mitochondrial DNA were filtered out (50). Altogether, 24,007 cells were kept for further analysis.

### Dimensionality Reduction

Dimensionality reduction was done using Seurat package in R. Following Seurat recommendations regarding the selection of variable genes, genes with mean expression higher than 0.0125 and lower than 2, and dispersion (log(variance/mean)) higher than 0.9 were analyzed using FindVariableGenes function. 2,399 variable genes were selected to create the principal components (PCs) of all cells. PCs were assembled using RunPCA function and their significance was calculated using JackStraw function. To create the t-SNE projections, the first 20 PCs (p<7.2E-18) were used via RunTSNE function by the elbow method, as suggested in Seurat tutorial. Perplexity of the t-SNE projection was set to 10, 30 (default), 50, 100 and 150, and showed similar shapes of projections. Thus, default perplexity was used.

Differential Gene Expression Analysis and Discriminating Markers between Cells from Young and Old Mice

Both analyses were carried via Seurat R package, using FindMarkers function. In the differential gene expression analysis, only highly and significantly expressed genes in young or old cells were counted as differentially expressed (|log(fold-change)|>0.2, adjusted p<10⁻³). In the discriminating markers analysis, markers of cells coming from each age group were calculated using receiver operating characteristic (ROC) analysis (test.use parameter was set to ‘roc’). To find strong discriminative markers, we set the expression fold-change threshold to a higher value (log(fold-change)>0.4).

Differential gene expression analysis between RECs and their closely-related, well-established subsets was done by using cells from old mice and using FindMarkers function. Only highly and significantly expressed genes were counted as differentially expressed (|log(fold-change)|>0.2, adjusted p<10⁻³).

### Clustering Process, Robustness Analysis and Subsets' Identity Association

Cells were clustered by applying the Seurat FindClusters function to the expression profiles of variable genes of all cells that passed quality control, projected on the first 20 PCs. The differences between the identified clusters were tested by applying the AssessNodes function to clusters' mean gene expression profile. Clusters that were indistinguishable from each other (out-of-bag error>0.05, random forest classifier) were merged.

To assess the robustness of the seven identified clusters, it was tested whether results differ upon selection of different numbers of PCs and different values for the resolution parameter. The clustering process was repeated based on the first 10, 19 and 21 PCs. These led to similar clustering patterns on the t-SNE projection. The resolution parameter was set to several values (0.6, 0.8 (default), 1 and 1.2, as recommended by Seurat). The different values resulted in varying numbers of clusters, yet clusters were then merged by the AssessNodes function to seven clusters with similar positions on the t-SNE projections.

To associate the clusters identified by the clustering process with previously reported CD4 T-cell subsets, differential expression analysis was applied to the expression profiles of cells. Differentially expressed genes in specific subsets were found using FindConservedMarkers function in Seurat, while accounting for the varying proportions of each age group per subset. Only highly and significantly expressed genes were counted as differentially expressed (|log(fold-change)|>0.2 and combined p<10⁻³). To associate each cluster with a potential identity, the top differentially expressed genes per cluster were manually compared to canonical markers of previously reported CD4 T-cell subsets.

### Regulons

Regulons were identified in SCENIC v0.1.6 package in R (36). Expression data pertaining to cells from old mice were used. Only genes with at least one raw count in at least one cell were analyzed throughout the pipeline. All gene raw counts were then log-transformed. Regulons were identified by SCENIC only if they were found to be active in more than 1% of all cells and correlated with at least one other regulon (|r|>0.3). In downstream analysis it was focused on high-confidence regulons, which were defined as active in more than 10% of the cells in at least one of the subsets in all mice and including high-confidence relationships between the transcription factor and at least nine target genes.

### Flow Cytometry

For extracellular staining, cells were washed with FACS staining buffer (PBS supplemented with 2% FBS and 1mM EDTA) and incubated with Fc receptor blocker (TrueStain fcX; BioLegend) for 5 minutes at 4° c. To differentiate between live and dead cells, a viability staining step was done using an eFluor780-Fixable Viability dye (eBioscience) following manufacture instructions. Cells were then incubated with primary antibodies for 25 minutes at 4° c and were washed twice with a FACS staining buffer. The following antibodies were used for membranal staining: PE-conjugated anti-CTLA4 (4C10-4B9; BioLegend), PE/cy7-conjugated anti-CD25 (3C7; BioLegend), AF700-conjugated anti-CD62L (Mel-14; BioLegend), BV605 or BV785-conjugated anti-PD1 (29F.1A12; BioLegend), APC-conjugated anti-CD81 (Eat-2; BioLegend), FITC-conjugated anti-CD8 (53-6.7; BioLegend), PerCP/cy5.5-conjugated anti-CD44 (IM7; BioLegend), AF700 or BV785-conjugated anti-CD4 (RM4-5; BioLegend), PE-conjugated anti-CD121b (4E2; BD Biosciences), BV421-conjugated anti-CD25 (PC61; BioLegend), BV605-conjugated anti-CD195 (C34-3448; BD Biosciences), BV785-conjugated anti-LAG3 (C9B7W; BioLegend), BV421-conjugated anti-CD4 (GK1.5; BioLegend), PerCP/cy5.5-conjugated anti-CD8 (53-6.7; BioLegend), PE-conjugated anti-CD137 (17B5; BioLegend), PE/cy7-conjugated anti-CD134 (OX-86; BioLegend) and PE-conjugated anti-CD178 (MFL3; eBioscience). After staining for membranal markers, intracellular labeling was performed: Cells were fixed and permeabilized using the FOXP3/Transcription factor staining kit (eBioscience), blocked with Rat serum (1 μl per 100 μl of staining buffer) and stained with the following antibodies: BV605-conjugated anti-TNF (MP6-XT22; BioLegend), BV605-conjugated anti-IL17a (TC11-18H10.1; BioLegend), FITC or BV510-conjugated anti-IL2 (JES6-5H4; BioLegend), BV421 or BV786-conjugated anti-IFN-γ (XMG1.2; BioLegend), BV421-conjugated anti-IL10 (JESS-16E3; BioLegend), APC-conjugated anti-Granzyme B (QA16A02; BioLegend), PE-conjugated anti-CCL5 (2E9/CCL5; BioLegend), PE/cy7-conjugated anti-EOMES (Dan 1 lmag; eBioscience), AF488-conjugated anti-FOXP3 (150D; BioLegend), APC-conjugated anti-IL21 (#149204; R&D systems), PE/dazzle-conjugated anti-Helios 22F6; Biolegend) and APC-conjugated anti-Perforin (B-D48; BioLegend). All flow cytometry experiments were performed with the CytoFLEX instrument (Beckman Coulter). Data were analyzed with the FlowJo (v-10.5.3) software. Gating strategies were set based on fluorescence minus one (FMO), unstained samples and unstimulated samples (when needed). All the samples in the experiment excluded dead cells, clumps and debris.

### Clustering Analysis of Flow Cytometry Data

Clustering analysis of flow cytometry data was done using FlowJo (v-10.5.3). First dead cells, doublets and non-lymphocyte cells were excluded (based on viability staining and FSC/SSC channels). CD4⁺ cells were used for further analysis. Data were sampled using “down sampling” function to get 40,000 representative cells from each sample. Then, a t-SNE algorithm was applied with the following parameters: Iteration=1000, Perplexity=40, Learning rate (eta)=2800. Mean fluorescence intensity (MFI) projected on the t-SNE plots for each protein to infer the clusters identity.

### Proliferation Assay and Cytokine Measurements

T cells were isolated from spleens of mice with a magnetic microbeads negative selection kit (EasySep™ Mouse CD4⁺ T Cell Isolation Kit; StemCell Technologies), and labeled with 5 μM carboxyfluorescein diacetate succinimidyl ester (CFSE; CellTrace™ proliferation kit, Invitrogen) for 5 minutes in PBS containing 5% fetal bovine serum, at room temperature. After incubation, cells were washed twice with PBS, re-suspended in RPMI-enriched medium (composition stated above) and activated with anti-CD3/anti-CD28 beads (Dynabeads™, Gibco) in 96-well (U shape) plates (80×10⁵ cells/well). After 72 hours, cells were collected and analyzed by flow cytometry.

Cytokine measurements: Isolated CD4⁺ T cells were incubated with anti-CD3/anti-CD28 beads for 48 hours. Six hours prior to analysis of TNF, IL-10, IFN-γ, granzyme B, perforin, IL-17a, IL-21, IL-2 and CCL5, cells were treated with Brefeldin A (GolgiPlug™, BD Biosciences), 1 μl per ml of cell culture (containing ˜10⁶ cells). The cells producing the cytokines were identified with flow cytometry (see Materials and methods, section “Flow cytometry”). Cells were cultured at 37° and 5% CO₂.

### Suppression Assay

For in vitro suppression assay, naïve CD4⁺ T cells were isolated from spleens of young (2 months) CD45.1 mice using naïve isolation kit (EasySep™ Mouse Naïve CD4⁺ T Cell Isolation Kit, StemCell Technologies), labelled with CFSE (CellTrace™ proliferation kit, Invitrogen) and used as responder cells (2×10⁴ cells per well). Then, cells were cultured in 96-well plates with irradiated 2×10⁴ APCs (as feeder cells) in the presence of sorted CD25^{high}CD81⁻ or CD25^{high}CD81⁺ Tregs at 1:1, 1:2 and 1:4 responders:Tregs ratios. Cells were stimulated with anti-CD3 (1 μg/ml) for 72 hours. Proliferation (defined as all cells with CFSE dilution) of responder cells was analyzed to assess the suppression of Tregs cells. The percentage of suppression was determined as (100−(% of proliferating cells with Tregs)/(% of proliferating cells without Tregs)).

### Serum Cytokines Measurement

Mouse peripheral blood was extracted after right atrial puncture into a 2 ml Eppendorf. Then, blood tubes were incubated at room temperature for coagulation (15 minutes). After incubation, tubes underwent centrifugation step (450 g), and serum was collected. For cytokines measurement, LEGENDplex mouse inflammation kit (BioLegend) was used following manufacture instruction. Data were acquired on CytoFLEX instrument (Beckman Coulter), and analyzed using LEGENDplex analysis software.

### Statistical Analysis for Flow Cytometry Experiments

Spearman correlation between the age of mice and the proportions of RECs and naïve cells in spleen was computed in R v3.4.2 using stats package v3.4.1. For statistical analysis GraphPad Prism (version 7.0a) was used. Paired T test was used for comparisons between two groups from the same biological samples. For analysis of more than two group, one-way ANOVA was used and corrected by Bonferroni correction for multiple comparisons.

### EXAMPLE 1

### scRNA-seq Reveals Complex Gene Expression Signature of CD4 T Cells in Aged Mice

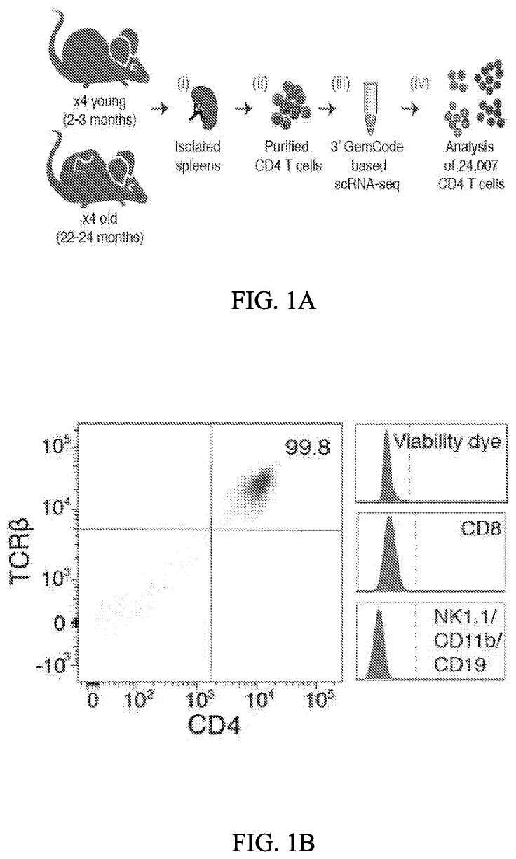

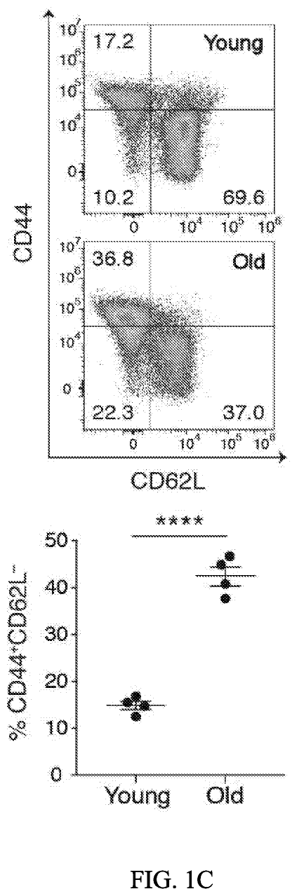

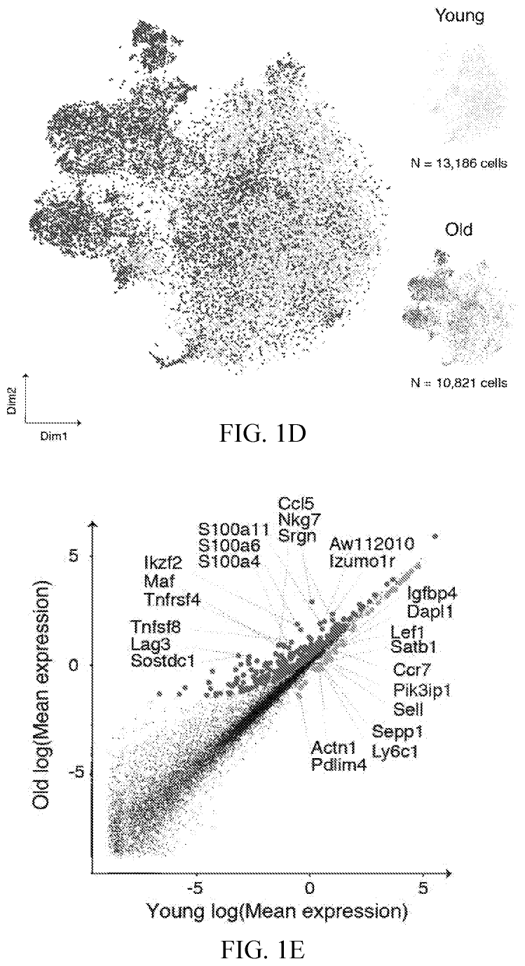

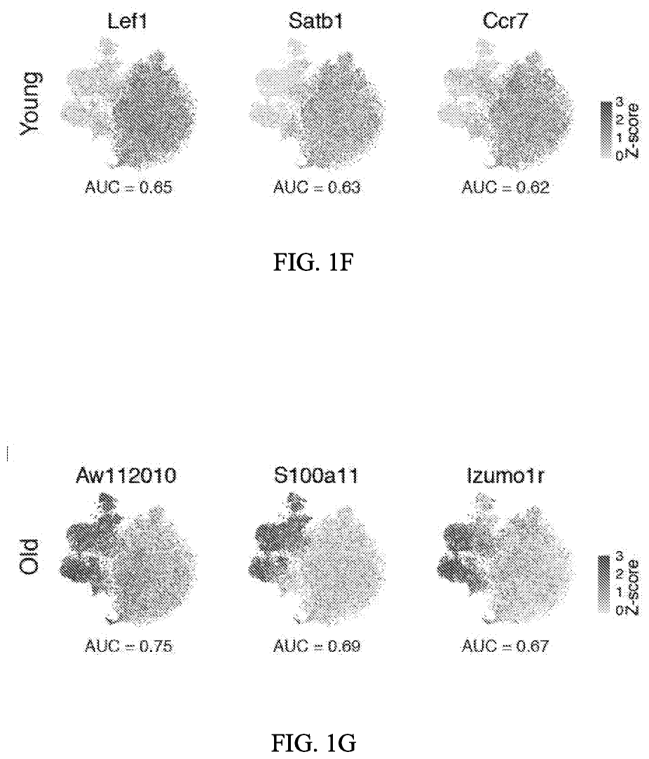

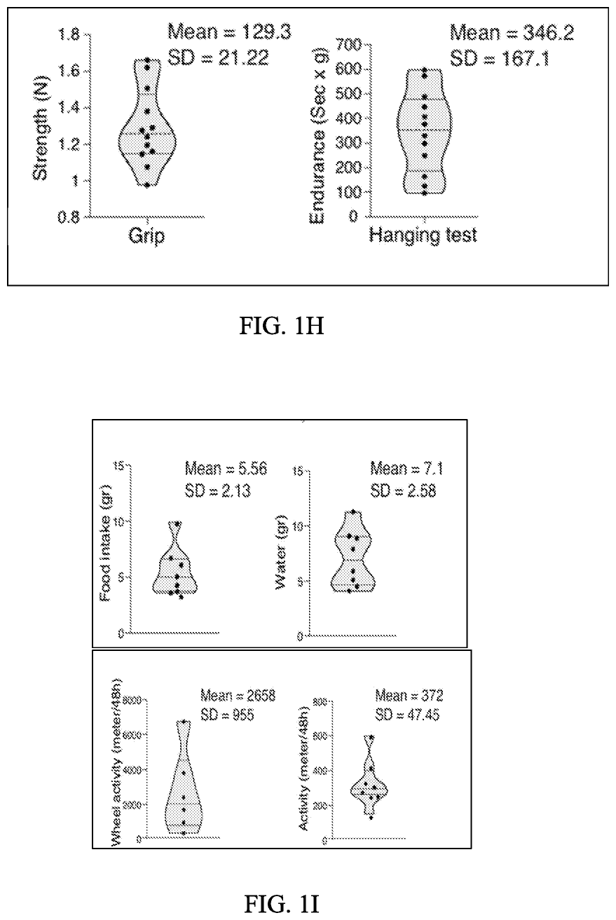

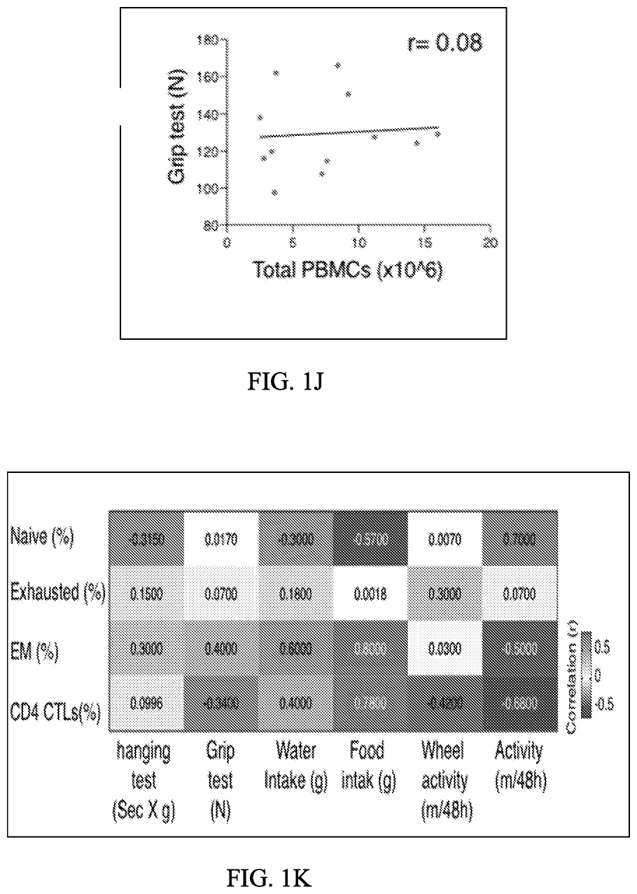

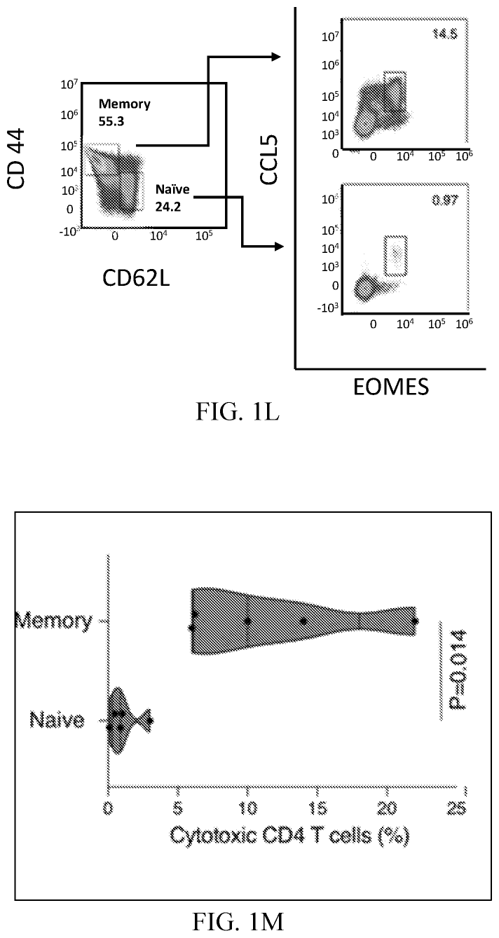

scRNA-seq data was generated from splenic CD4 T cells of young (2-3 months; n=4) and old (22-24 months; n=4) healthy mice, henceforth denoted young and old cells, respectively. The experimental procedure is shown in FIG. 1A. In brief, (i) splenocytes were harvested from young (2-3 months, n=4) and old (22-24 months, n=4) mice; (ii) CD4 T cells were purified using magnetic separation and sorting; (iii) cells' mRNAs were barcoded using 10× Genomics Chromium platform and sequenced; and (iv) data were computationally analyzed (FIG. 1A).

Cells were subjected to two rounds of CD4 enrichment followed by sorting for CD4+TCRb+CD8− CD19-CD11b-NK1.1− cells, to achieve highly pure (<99%) CD4 T cells (FIG. 1B) suitable for use in the scRNA-seq experiments. To assess the gross shift of CD4 T cells from naïve to memory phenotype, canonical surface markers were measured using flow cytometry. As expected, a significant reduction in the abundance of naïve cells (CD4+CD62L+CD44−) and an increase in the frequency of effector-memory cells (CD4+CD44+CD62L−) in the old versus the young splenic CD4 T cells was observed (FIG. 1C). The upper panel of FIG. 1C shows representative flow cytometry plots of cells from young and old mice and the lower panel represents cells from old mice showing a shift toward effector-memory identity. The data in FIG. 1C was obtained from two different experiments (n=2 in each age group per experiment), where each dot represents a mouse, and bars represent mean ±SEM (unpaired T test, ****p<10⁻⁴). In addition, the majority of cytotoxic CD4 T cells in old mice have an effector memory phenotype: CD44+CD62L− (FIGS. 1L and 1M).

Next, thousands of these cells were sequenced using the 10x GemCode Chromium platform (18) to explore the transcriptomic differences between the age groups. Expression profiles of 13,186 and 10,821 CD4 T cells from young and old mice, respectively were obtained (data not shown).

Subsequently, dimensionality reduction was applied to the expression profiles. For this, genes with variable expressions were selected, projected on the first 20 principal components, followed by a t-distributed stochastic neighbor embedding (t-SNE). Cells did not show a topological gathering by individual mouse, depth of sequencing or experimental batch (data not shown). Surprisingly, old cells grouped together in a distinctive manner, thus highlighting the impact of aging on the transcriptome of CD4 T cells (FIG. 1D). Differential expression analysis revealed that while genes upregulated in young cells (log(fold-change)>0.2, adjusted p<10-3) were associated with a naïve phenotype (e.g., Lef1, Ccr7 and Sell genes), genes upregulated in old cells presented a mix of inflammatory (e.g., S100a4, S100a11 and Cc15 genes) and regulatory (e.g., Maf, Ikzf2 and Tnfrsf4 genes) signatures, as well as exhaustion markers (e.g., Lag3 and Tnfsf8 genes; FIG. 1E). In contrast to the relatively homogeneous signature of young cells (turquoise/gray dots, right side of the major trend shown in FIG. 1E), the highly expressed inflammatory, regulatory, and exhaustion genes in old cells (brown black dots, right side of the major trend shown in FIG. 1E) suggest an intricacy of the CD4 T-cell compartment in old mice. Shared genes exclusively expressed in old or young cells were identified using receiver operating characteristic (ROC) analysis, while focusing on genes that were commonly and highly expressed in each age group. As seen in FIG. 1F, Lef 1, Satb1 and Ccr7 genes were the top three markers common to young cells (AUC>0.61, power>0.23), supporting the dominancy of naïve CD4 T cells in young age. The three top markers common to old cells were the genes Aw112010, S100a11 and Izumo1r (AUC>0.66, power>0.33; FIG. 1G), which were reported to be upregulated under chronic inflammatory conditions. These genes imply a strong relationship between the inflammatory state and the identity of CD4 T cells in aging and can be used as general markers for aged CD4 T cells.

Old mice also exhibit a marked variability in physical and metabolic biomarkers of aging (FIGS. 1H-1I). While no correlation was observed between the total number of PBMCs and physical activity (FIG. 1J), the CD4 CTLs were positively correlated with food and water intake, and inversely correlated with physical activity. The results are summarized in the heatmap presented in FIG. 1K. Overall, these data raise the possibility that high frequencies of CD4 CTLs are associated with a trajectory of physiological and inflammatory settings that may increase the rate of aging.

### EXAMPLE 2

### CD4 T Cells Undergo Extensive Diversification with Age, Resulting in a New Population Structure

In order to classify CD4 T-cell subsets in an unbiased manner, cells were clustered by their transcriptomic profiles and the robustness of the clusters' identity assessed. Seven distinct and robust clusters were identified (FIG. 2A). To associate each cluster with a known CD4 T-cell subset, the most significantly upregulated genes (combined p<10-3) of each cluster were screened and compared to previously reported CD4 T-cell subsets and to canonical markers (FIG. 2B and FIG. 2C). Specifically, all CD4 T cells were grouped by subset and age (FIG. 2B - horizontal bars). Genes shown were upregulated significantly (log(fold-change)>0.2, combined p<10⁻³) in at least one subset compared to all other cells. Genes were ordered by significance and associated with the subset with higher detection rates. Violin plots presenting the expression (z-score) were formed for the following selected canonical marker genes across all seven subsets: Left, FOXP3, Lgals1, Lag3, Gzmk, Sell, IL2RA, S100a4, Pdcd1, Eomes, Igfbp4, Ikzf2, Itgb1, Tnfrsf8 and Ctla2a (FIG. 2C).

Of the seven distinct clusters, four were matching established subsets: two populations of naïve T cells overexpressing Sell, Lefl and Igfbp4 genes, which differ by the expression of Isg15 gene (denoted naïve and naïve_Isg15); a population of resting regulatory T cells (rTregs), labeled based on their classical expression of Foxp3 and Il2ra genes, together with the expression of naïve-associated genes Lef1 and Sel1; and effector-memory T cells (TEM) expressing the S100a4, Igals1 and Itgb1 genes. The transcriptional signatures of the three remaining subsets have not been previously defined in the context of aging, and include: activated regulatory T cells (aTregs) overexpressing Foxp3, Cd81, Cd74 and Cst7 genes, together with aTregs-associated genes such as Tnfrsf4, Tnfrsf9, Tnfrsf18 and Ikzf2 genes; cells with an exhaustion signature (denoted exhausted) overexpressing the Lag3, Tbc1d4, Sostdcl and Tnfsf8 genes; and cells overexpressing genes such as Eomes, Gzmk and Ctla2a, which are commonly associated with CD8 T cells (denoted cytotoxic), and were previously described in the context of viral infection and cancer as CD4 cytotoxic T cells.

Next, the proportion of each subset in old versus young mice was compared and is presented in Table 1, below and further illustrated in FIG. 2D.

TABLE 1
Proportions of cellular subsets in a young mouse and an old mouse.

| Cell subset | Presence in young mice (%) | Presence in old mice (%) |
|---|---|---|
| Cytotoxic | 0.1 | 9 |
| TEM | 4.8 | 14.9 |
| Exhausted | 0.4 | 12.7 |
| aTregs | 0.9 | 9 |
| rTregs | 3.1 | 3.2 |
| Naïve_Isg15 | 4.7 | 3.2 |
| Naïve | 86 | 48 |

Enrichment was computed as the log odds ratio between the frequency of each subset in old versus young mice, across all pairs (FIG. 2E). Naive subsets were enriched in young mice (Naive: log(median)=−0.27, p=0.03, and Naive_Isg15: log(median)=-0.23, p=0.03). rTregs subset was equally distributed (log(median)=0.02, p=0.89). Four subsets were enriched in every old mouse: TEM (log(median)=0.51, p=0.03), aTregs (log(median)=1, p=0.03), exhausted (log(median)=1.32, p=0.03) and cytotoxic (log(median)=1.46, p=0.03) subsets. Whereas the two naïve subsets were significantly enriched in young mice, the rTregs subset had a similar abundance in both age groups, while the TEM subset was dominant in old mice. Notably, the aTregs, exhausted, and cytotoxic subsets (collectively denoted RECs to represent these Regulatory, Exhausted and Cytotoxic subsets) were highly enriched in all aged mice, accounting for ˜30% of the CD4 T cells and were negligible in young mice (˜1%).

Since the frequency of RECs substantially differed between old mice (FIG. 2E) it was examined whether this pattern represents different stages of chronic inflammation. Accordingly, the level of serum cytokines in the young and the old mice were measured and correlated with the proportion of RECs in each mouse (FIG. 2F). Whereas all cytokines and chemokines measured had the highest positive correlation with the abundance of the cytotoxic subset of CD4 T cells, and significant correlation with all subsets of RECs.

Overall, the results demonstrate that aging is marked by a complex landscape of CD4 T cells, with expansion of subsets with effector (including TEM, exhausted and cytotoxic cells) and regulatory (aTregs) signatures, associated with serum markers of chronic inflammation. A schematic illustration of the major changes that occur in the population of CD4 T cells during aging, is shown in FIG. 2G, demonstrating the shift from naïve dominancy in young mice to diverse and extreme effector and regulatory phenotypes in old mice

### EXAMPLE 3

### RECs are Distinct CD4 T-Cell Subsets that Gradually Accumulate with Age

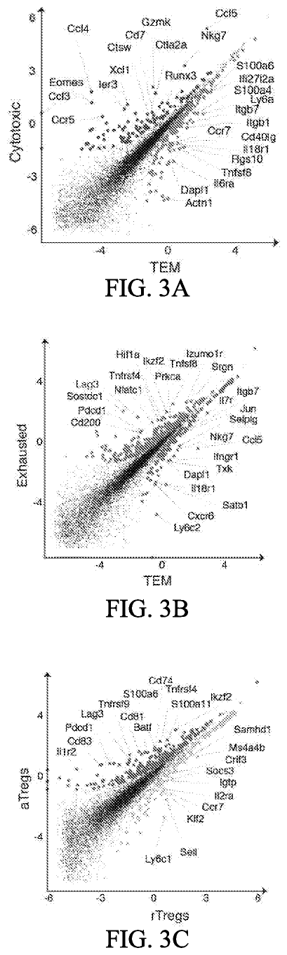

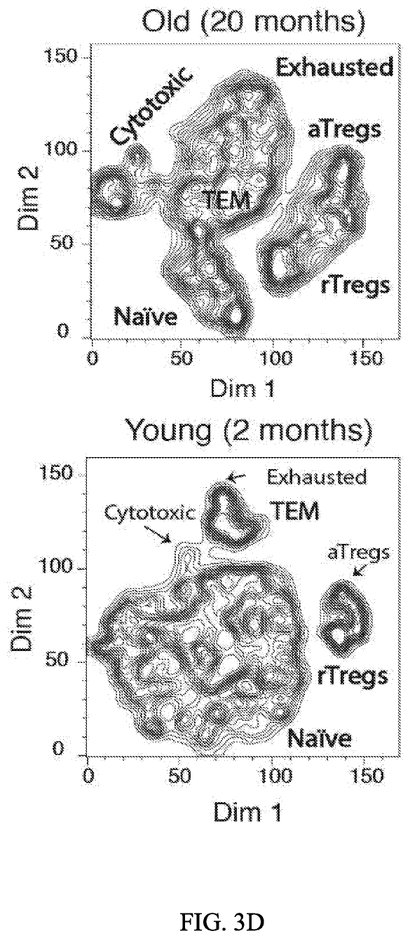

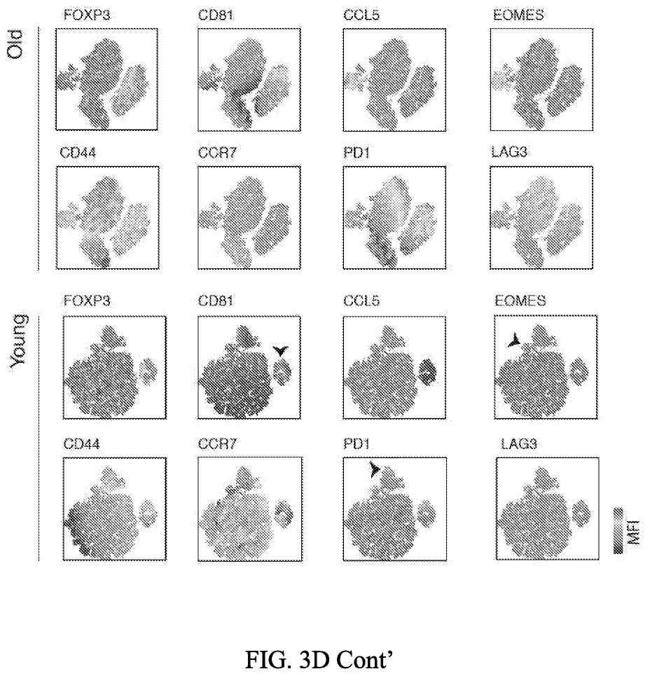

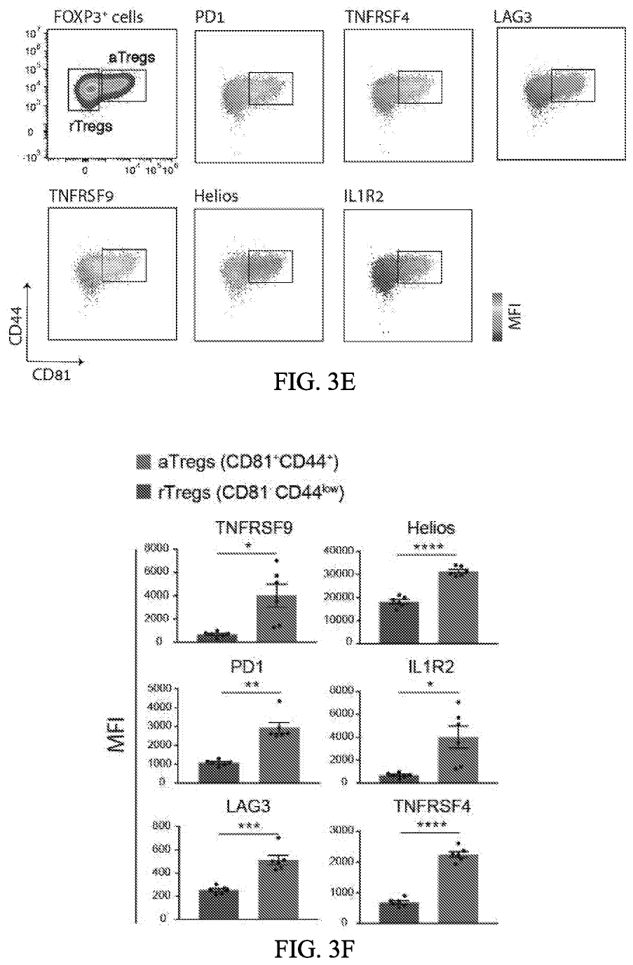

To gain insight into the identity of RECs, the expression profiles to the profiles of RECs were compared to closely-related, well-established subsets. First, the cytotoxic and exhausted cells were compared to TEMs (FIGS. 3A and 3B): The cytotoxic cells exhibited higher levels of Nkg7, Runx3, Eomes and Gzmk genes, together with genes encoding inflammatory chemokines (e.g., Cc13, Cc14, Cc15 (FIG. 3A). The exhausted cells expressed relatively high levels of co-inhibitory genes (e.g., Cd200, Lag3 and Pdcd1), as well as genes related to cellular stress (e.g., Hif1a), indicating a dysfunctional phenotype (FIG. 3B); The TEM subset expressed higher levels of memory associated genes (e.g., Itgb7 and IL-18R1) in both comparisons. Next, the gene expression profiles of the two Tregs subsets were compared (FIG. 3C). Notably, whereas the rTregs subset expressed higher levels of quiescence genes (e.g., Ms4a4b and Sell), aTregs subset expressed higher levels of co-inhibitory genes (e.g., Pdcd1 and Lag3), co-stimulatory genes (e.g., TNFRSF9 and TNFRSF4) and activation genes (S100a11, IL1R2 (IL1 decoy receptor) and Ikzf2.

The identities of the subsets were further validated by measuring the levels of key surface and intracellular proteins (chosen based on the gene expression profiles) in CD4 T cells from young (2 months) and old (20 months) mice, using multicolor flow cytometry. Clustering analysis revealed that whereas the young cells comprised mainly naïve cells and two defined clusters of effector memory and rTregs cells, the old cells comprised a prominent cytotoxic CD4 T-cell subset, expressing high levels of the EOMES transcription factor (TF) and the CCL5 chemokine (FIG. 3D, left panels). Naïve, TEM, exhausted (based on the expression of CCR7, CD44, LAG3 and PD1) and the two Tregs subsets (all were FOXP3+ and differed by the expression levels of CD81, PD1 and LAG3; FIG. 3D) were also identified. Further analysis of the Tregs cluster in the old cells revealed that CD81, together with CD44, distinguish the aTregs from all FOXP3+ cells. These markers were co-expressed with high levels of PD1, TNFRSF4, TNFRSF9, Helios, IL1R2 and LAG3, which together affirmed their activated identity (FIG. 3E and FIG. 3F).

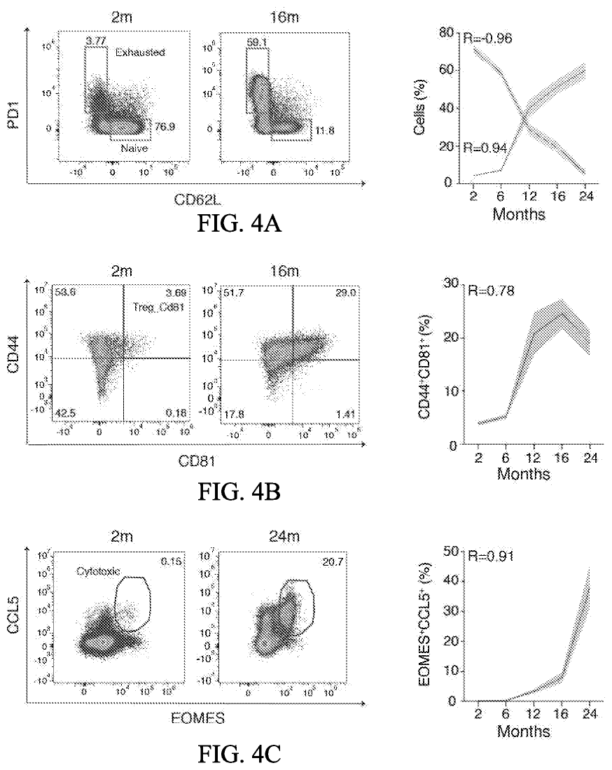

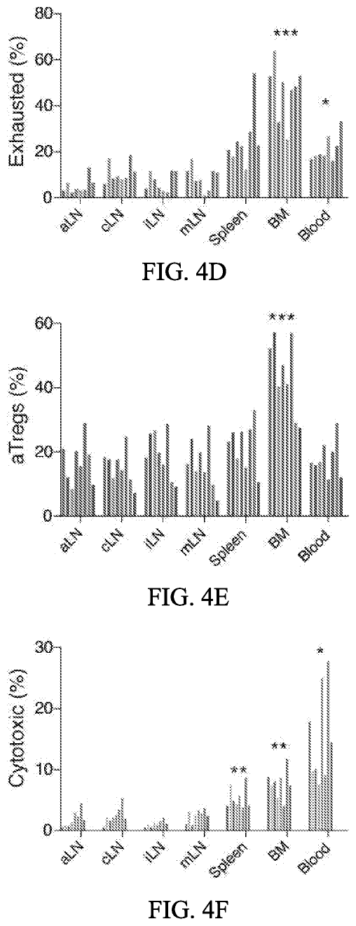

To assess the dynamics of RECs over time, their relative abundance in spleens of healthy mice at 2, 6, 12, 16 and 24 months of age, was measured using flow cytometry. Exhausted cells (defined as CD4+PD1+CD62L−FOXP3−EOMES−CCL5−), steadily accumulated from 6 months of age (r=0.94, p=1.5×10-12, Spearman correlation), and coincided with continuous decreased proportions of naïve cells (defined as CD4+CD62L+PD1−CD81−EOMES−CCL5−; r=−0.96, p=1.7×10-14, Spearman correlation; FIG. 4A). Out of the regulatory cells (CD4+FOXP3+), the relative abundance of aTregs, (defined as CD81+CD44+) also increased with age, reaching a peak at 16 months of age and slightly declining at 24 months (r=0.78, p=10-8, Spearman correlation; FIG. 4B). Cytotoxic cells (defined as CD4+EOMES+CCL5+CD8−), were observed at a later time point at 12 months, and their fraction sharply increased with age (r=0.91, p=2.2×10-16, Spearman correlation; FIG. 4C). Gating in this analysis was based on unstained samples and fluorescence minus one (FMO). Data include three (FIGS. 4B and 4C) or two (FIG. 4A) different experiments, and n=5-10 per time point.

Since the cellular composition of the spleen is different from other immunological tissues, and dynamically change with aging, site-specific proportions of RECs were also examined. For this purpose, CD4 T cells were purified from seven immune compartments of 16 months-old mice, including spleen, bone marrow (BM), blood and lymph nodes (LNs) from axillary, cervical, inguinal and mesenteric sites, and the percentage of RECs in each compartment was analyzed by flow cytometry. Whereas all RECs showed the lowest proportion in the LNs, they were highly abundant in BM, in particular aTregs (FIG. 4D-FIG. 4F). Additionally, the exhausted and cytotoxic cells were enriched in blood and spleen. Together, these findings indicate that the RECs' accumulation with time predominantly occurs in non-lymphatics immune sites, opposed to lymphatic sites, which present relatively “juvenile signature” of T-cell composition.

### EXAMPLE 4

### RECs Exhibit Extreme Regulatory and Effector Properties

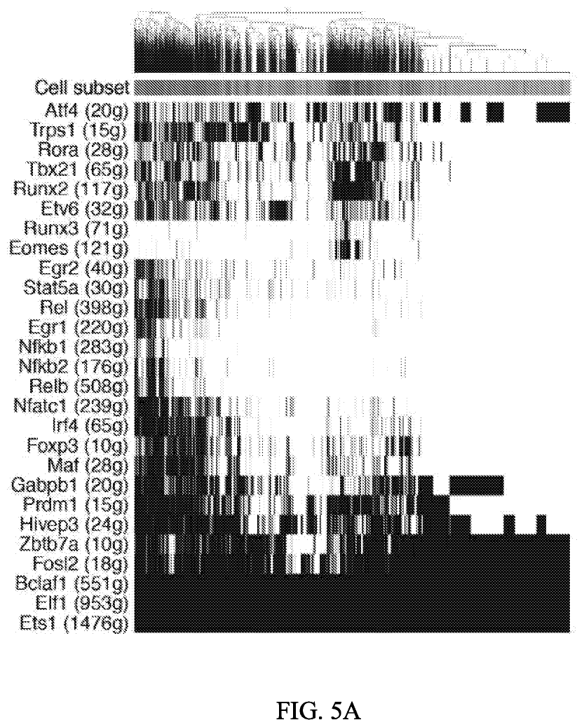

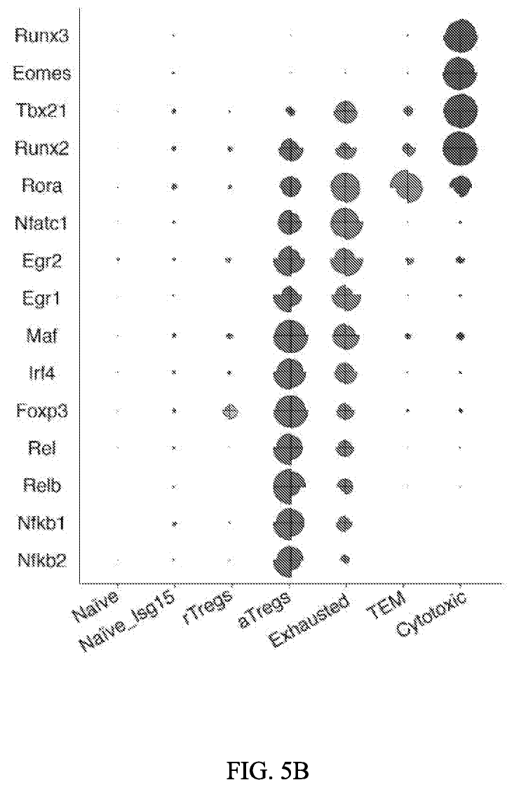

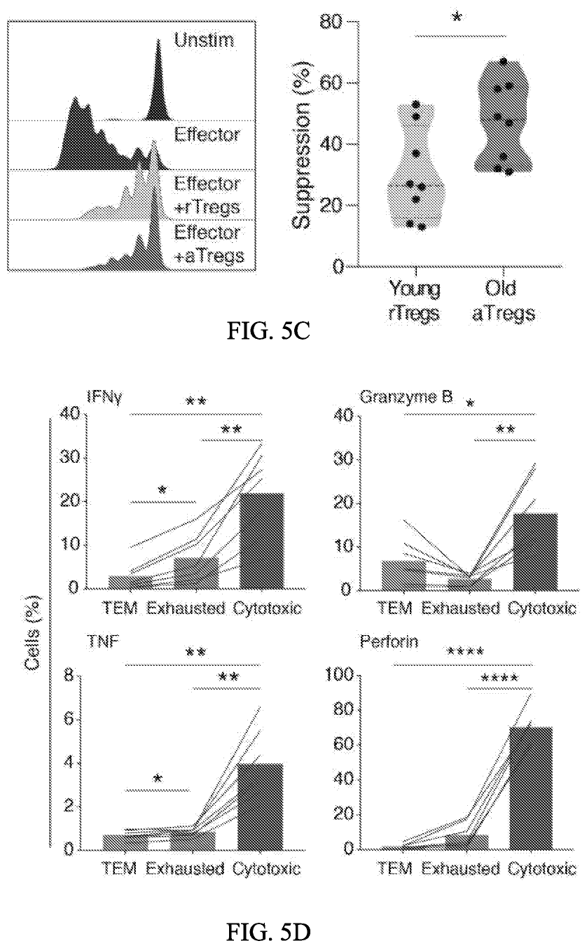

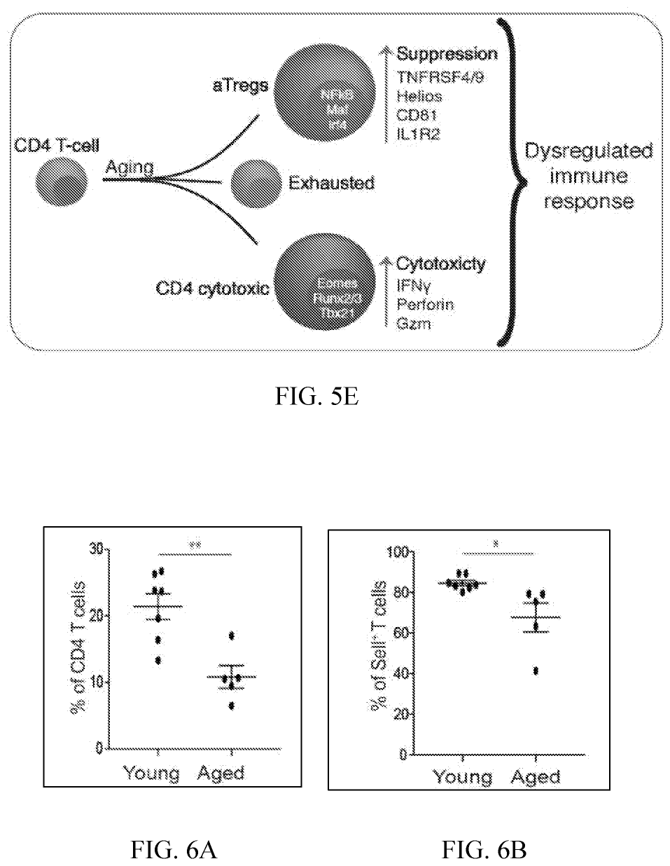

As the regulation of CD4 T-cell identity and function is largely coordinated by the combined activity of TFs, the specific set of working regulons (namely TFs and their target gene modules) that appear to be active within the identified subsets and specifically within the RECs were evaluated. For this, the SCENIC workflow was applied on the transcriptional signatures of the old cells. 27 high-confidence regulons that were active consistently across all four old mice were identified (FIG. 5A). Clustering cells hierarchically by their active regulons revealed that cells were largely grouped by the subsets identified. The three regulons that were active in all subsets were governed by the TFs Bclaf1, Elf1 and Ets1, previously shown to be crucial for peripheral T-cell homeostasis and activation. The aTregs subset displayed elevated activity of the regulons corresponding to Foxp3, NFKB TF family (Nfkb1, Nfkb2, Rel and Relb), Irf4 and Maf, which were previously associated with activation phenotype of Tregs; FIG. 5B). To assess whether the two Tregs subsets differ in functionality, aTregs (CD25highCD81+; FIG. 5C: brown) from old mice (16 months) were sorted and their suppressive function was compared to that of rTregs (CD25highCD81−; FIG. 5C: yellow) isolated from young (2 months) mice. Suppressive function was assessed ex-vivo after 72 hours of co-culture with activated naïve CD4 T cells from young CD45.1 mice. The reduction in the proliferation of the activated CD4 T cells was measured via flow cytometry and calculated as % of suppression. Indeed, aTregs exhibited significantly higher suppressive activity than rTregs ex-vivo (FIG. 5C).

The cytotoxic subset seemed to be dominantly regulated by TFs associated with cytotoxicity, including Eomes, Runx2, Runx3 and Tbx21 regulons (FIG. 5B). In activated CD8 T cells, these TFs regulate the production of cytokines and lytic proteins, such as tumor necrosis factor (TNF), interferon-gamma (IFNγ), granzyme B (GzmB) and perforin. To evaluate the activity of these regulons in aged cytotoxic CD4 T cells, subset-specific secretion of these molecules was measured from activated CD4 T cells extracted from 16-months old mice. Compared to exhausted and TEM subsets, the cytotoxic subset showed higher production of TNF, IFNγ, perforin and GzmB, indicating its active cytotoxic capability and pro-inflammatory function, following stimulation (FIG. 5D). A higher production of IL21 and IL17A in cytotoxic cells was also observed, probably due to the active Rora regulon (FIG. 5B). Exhausted cells presented a high activity of Nfatc1 regulon, which is probably involved in regulating the exhaustion process.

Taken together, these results demonstrate that each of the three RECs employs defined sets of genes and TFs, which together promote an extreme immune phenotype that can hinder immunity in old age (FIG. 5E).

### EXAMPLE 5

### Clinical Studies—Healthy Elderly Individuals Accumulate Cytotoxic CD4 T Cells

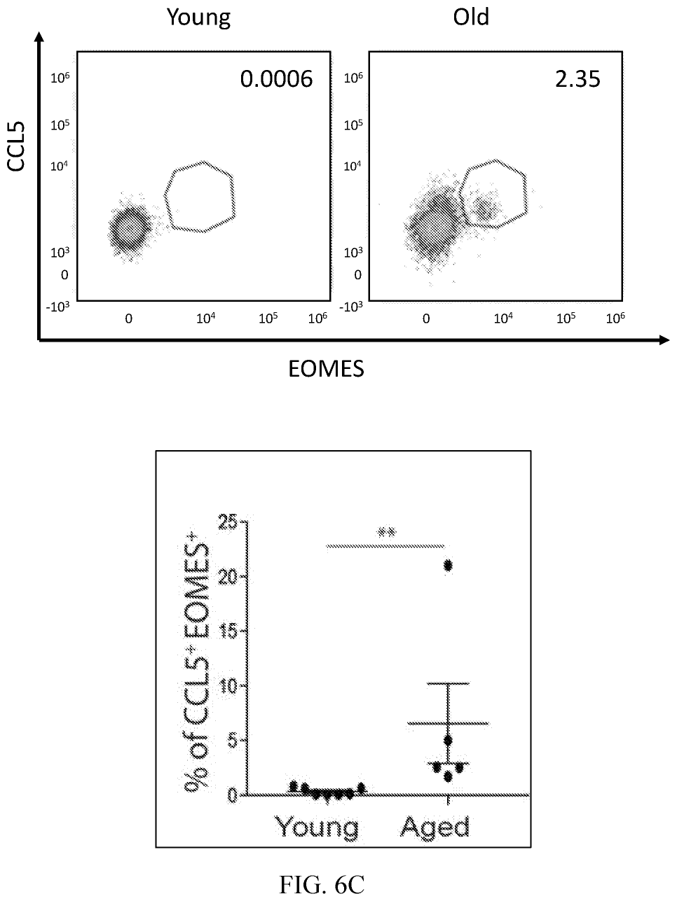

A preliminary clinical study in human subjects revealed that CD4 CTLs accumulate also in elderly, but not in adult, healthy human individuals, as shown in FIGS. 6A-6C and detailed below.

Blood PBMCs was obtained from healthy young (age: 25-35 years, n=7) and old (age: >70 years, n=5) human individuals and analyzed by flow cytometry. As shown in FIGS. 6A and 6B, the frequency of total CD4 T (FIG. 6A) and naïve CD4 T (FIG. 6B) cells was decreased in old compared with young PBMCs.

In addition, the frequency of CD4 CTLs is higher in elderly than in young human individuals, where in young individual these cells are barely detected (FIG. 6C, t-test, *p<0.05, **p<0.01).

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-73. (canceled)

74. A method of evaluating an immunological age of an immune system of a subject, the method comprising:
a) obtaining a biological sample from a subject, the biological sample comprising biomarkers associated with one or more subsets of CD4 T cells;
b) detecting a level of a plurality of the biomarkers, thereby identifying the presence of one or more subsets of CD4 T cells in the immune system, wherein the one or more subsets of CD4 T cells comprise at least one of cytotoxic T cells and activated regulatory (aTreg) CD4 T cells; and
c) evaluating the immunological age of the immune system based on the presence of the plurality of biomarkers identified in the biological sample.

75. The method of claim 74, wherein the one or more subsets of CD4 T cells further comprise at least one of naïve CD4 T cells, naïve_Isg15 CD4 T cells, effector memory (TEM) CD4 T cells, exhausted CD4 T cell and, rTregs CD4 T cells.

76. The method of claim 74, wherein the one or more subsets of CD4 T cells is naïve CD4 T cells and wherein when the presence of the naïve CD4 T cells subset is above 50%, the immunological age of the immune system is young and when the presence of the naïve CD4 T cells subset is below 50%, the immunological age of the immune system is mature.

77. The method of claim 74, wherein the one or more subsets of CD4 T cells is cytotoxic CD4 T cells and wherein when the presence of the cytotoxic CD4 T cells subset is above 1%, the immunological age of the immune system is mature, and when the presence of the exhausted CD4 T cells subset is above 1%, thereby indicating that the immunological age of the immune system is mature.

78. The method of claim 74, wherein the one or more subsets of CD4 T cells is aTregs CD4 T cells and the presence of the aTregs CD4 T cells subset is above 1%, thereby indicating that the immunological age of the immune system is mature.

79. The method of claim 74, wherein the plurality of biomarkers comprises at least three biomarkers.

80. The method of claim 74, wherein the plurality of biomarkers is selected from CD137, CD134, FOXP3+, GITR+, Helios+, CD74, HLA-DR, and wherein the one or more subsets of CD4 T cells identified in the biological sample is aTreg CD4 T cell.

81. The method of claim 74, wherein the plurality of biomarkers is selected from Nkg7, Runx3, Eomes, Gzmk, IFN-b, IL-27, IL21, IL 17A, Cc13, Cc14 and Cc15, and wherein the one or more subsets of CD4 T cells identified in the biological sample is CD4 cytotoxic cells.

82. The method of claim 74, wherein the plurality of biomarkers is selected from Cd200, Lag3, Hif1a, Nfatc1 and Pdcd1, and wherein the one or more subsets of CD4 T cells identified in the biological sample is exhausted CD4 T cells.

83. A kit for evaluating an immunological age of an immune system of a subject, the kit comprising:
at least two biomarker identifiers configured to determine a level of at least two biomarkers in a biological sample, wherein the at least two biomarkers are associated with one or more subsets of CD4 T cells, and wherein the one or more subsets of CD4 T cells comprise CD4 cytotoxic cells and/or activated regulatory (aTreg) CD4 T cells; and
instructions for use.

84. A method of treating an age-related frailty in a subject in need thereof, the method comprising:
obtaining a biological sample from a subject comprising biomarkers associated with one or more subsets of CD4 T cells;
detecting a level of a plurality of the biomarkers thereby identifying the presence of one or more subsets of CD4 T cells in the immune system, wherein the one or more subsets of CD4 T cells comprise at least one of CD4 cytotoxic cells and activated regulatory (aTreg) CD4 T cells;
identifying an immunological age of the immune system based on the presence of the plurality of biomarkers identified in the biological sample; and
administering to the subject one or more therapeutics, based on the identified immunological age of the immune system.

85. The method of claim 84, wherein the one or more subsets of CD4 T cells further comprise at least one of naïve CD4 T cells, naïve_Isg15 CD4 T cells, effector memory (TEM) CD4 T cells, exhausted CD4 T cell and, rTregs CD4 T cells.

86. The method of claim 84, wherein one or more subsets of CD4 T cells is naïve CD4 T cells and wherein when the presence of the naïve CD4 T cells subset is above 50%, the immunological age of the immune system is young and when the presence of the naïve CD4 T cells subset is below 50%, the immunological age of the immune system is mature.

87. The method of claim 84, wherein the one or more subsets of CD4 T cells is cytotoxic CD4 T cells and wherein when the presence of the cytotoxic CD4 T cells subset is above 1%, the immunological age of the immune system is mature and when the presence of the exhausted CD4 T cells subset is above 1%, the immunological age of the immune system is mature.

88. The method of claim 84, wherein the one or more subsets of CD4 T cells is aTregs CD4 T cells and the presence of the aTregs CD4 T cells subset is above 1%, thereby indicating that the immunological age of the immune system is mature.

89. The method of claim 84, wherein the one or more therapeutics is selected from the group consisting of an antibody, a siRNA, a microRNA, a small molecule or any combination thereof and wherein the antibody is an NKG2D antibody, a CD7 antibody, a CD134 antibody, a CD137 antibody, a GITR antibody or any combination thereof

90. The method of claim 84, wherein the one or more therapeutics is an age-related drug.

91. The method of claim 90, wherein the age-related drug is a statin, an aspirin or a combination thereof

92. The method of claim 84, wherein the age-related frailty is chronic inflammation or cancer.

93. The method of claim 84, wherein the wherein the age-related frailty disease is immune deficiency.
